SEQUENCE LISTING (63 sequences)

SEQ ID NO: 1 - protein, 5 aa, Artificial Sequence
  synthetic peptide
YFLRR

SEQ ID NO: 2 - protein, 6 aa, Artificial Sequence
  synthetic peptide
  MISC_FEATURE (2)..(2): Xaa = Sarcosine
YXFLRR

SEQ ID NO: 3 - protein, 6 aa, Artificial Sequence
  synthetic peptide
  MISC_FEATURE (2)..(2): Xaa - Sarcosine
  MISC_FEATURE (6)..(6): Xaa - DArg
YXFLRX

SEQ ID NO: 4 - protein, 6 aa, Artificial Sequence
  synthetic peptide
  MISC_FEATURE (2)..(2): Xaa - Sarcosine
  MISC_FEATURE (3)..(3): Xaa - 4-nitro-phenylalanine
  MISC_FEATURE (6)..(6): Xaa - DArg
YXXLRX

SEQ ID NO: 5 - protein, 6 aa, Artificial Sequence
  synthetic peptide
  MISC_FEATURE (2)..(2): Xaa - Sarcosine
  MISC_FEATURE (3)..(3): Xaa - 4-nitro-phenylalanine
  MISC_FEATURE (5)..(5): Xaa - DArg
YXXLXR

SEQ ID NO: 6 - protein, 6 aa, Artificial Sequence
  synthetic peptide
  MISC_FEATURE (2)..(2): Xaa - Sarcosine
  MISC_FEATURE (5)..(5): Xaa - DArg
YXFLXR

SEQ ID NO: 7 - protein, 6 aa, Artificial Sequence
  synthetic peptide
  MISC_FEATURE (2)..(2): Xaa - Sarcosine
  MISC_FEATURE (5)..(6): Xaa - DArg
YXFLXX

SEQ ID NO: 8 - protein, 6 aa, Artificial Sequence
  synthetic peptide
  MISC_FEATURE (3)..(3): Xaa - 4-chloro-phenylalanine
  MISC_FEATURE (5)..(6): Xaa - DArg
YGXLXX

SEQ ID NO: 9 - protein, 6 aa, Artificial Sequence
  synthetic peptide
  MISC_FEATURE (2)..(2): Xaa - Sarcosine
  MISC_FEATURE (3)..(3): Xaa - 4-nitro-phenylalanine
  MISC_FEATURE (5)..(6): Xaa - DArg
FXXLXX

SEQ ID NO: 10 - protein, 6 aa, Artificial Sequence
  synthetic peptide
  MISC_FEATURE (2)..(2): Xaa - Sarcosine
  MISC_FEATURE (3)..(3): Xaa - 4-nitro-phenylalanine
  MISC_FEATURE (5)..(5): Xaa - DLys
  MISC_FEATURE (6)..(6): Xaa - DArg
YXXLXX

SEQ ID NO: 11 - protein, 6 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - DArg
YXXLRX

SEQ ID NO: 12 - protein, 6 aa, Artificial Sequence
  synthetic peptide
  MISC_FEATURE (2)..(2): Xaa - Sarcosine
  MISC_FEATURE (3)..(3): Xaa - 4-fluoro-phenylalanine
  MISC_FEATURE (6)..(6): Xaa - DArg
YXXLRX

SEQ ID NO: 13 - protein, 6 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (5)..(5): Xaa - N-methylarginine
  MISC_FEATURE (6)..(6): Xaa - DArg
YXXLXX

SEQ ID NO: 14 - protein, 6 aa, Artificial Sequence
  synthetic peptide
  MISC_FEATURE (2)..(2): Xaa - Sarcosine
  MISC_FEATURE (3)..(3): Xaa = 4-fluoro-phenylalanine
  MISC_FEATURE (5)..(5): Xaa - N-methylarginine
  MISC_FEATURE (6)..(6): Xaa - DArg
YXXLXX

SEQ ID NO: 15 - protein, 6 aa, Artificial Sequence
  synthetic peptide
  MISC_FEATURE (2)..(2): Xaa - Sarcosine
  MISC_FEATURE (3)..(3): Xaa - 4-nitro-phenylalanine
  MISC_FEATURE (5)..(5): Xaa - N-methylarginine
  MISC_FEATURE (6)..(6): Xaa - DArg
YXXLXX

SEQ ID NO: 16 - protein, 6 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - DArg
YXXLKX

SEQ ID NO: 17 - protein, 6 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - N-methylarginine
YXXLRX

SEQ ID NO: 18 - protein, 6 aa, Artificial Sequence
  synthetic peptide
  MISC_FEATURE (2)..(2): Xaa - Sarcosine
  MISC_FEATURE (3)..(3): Xaa - 4-nitro-phenylalanine
  MISC_FEATURE (6)..(6): Xaa - N-methylarginine
YXXLRX

SEQ ID NO: 19 - protein, 6 aa, Artificial Sequence
  synthetic peptide
  MISC_FEATURE (2)..(2): Xaa - Sarcosine
  MISC_FEATURE (3)..(3): Xaa - 4-nitro-phenylalanine
  MISC_FEATURE (5)..(6): Xaa - N-methylarginine
YXXLXX

SEQ ID NO: 20 - protein, 8 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - DArg
YXXLRXIR

SEQ ID NO: 21 - protein, 6 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - DArg
YXXLRX

SEQ ID NO: 22 - protein, 10 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - DArg
YXXLRXIRPK

SEQ ID NO: 23 - protein, 10 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - DArg
  MISC_FEATURE (8)..(8): Xaa - DArg
  MISC_FEATURE (10)..(10): Xaa - DLys
YXXLRXIXPX

SEQ ID NO: 24 - protein, 6 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - DArg
YXXLRX

SEQ ID NO: 25 - protein, 8 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - DArg
  MISC_FEATURE (8)..(8): Xaa - DArg
YXXLRXIX

SEQ ID NO: 26 - protein, 8 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - N-methylarginine
YXXLRXIR

SEQ ID NO: 27 - protein, 8 aa, Artificial Sequence
  synthetic peptide
  MISC_FEATURE (2)..(2): Xaa - Sarcosine
  MISC_FEATURE (3)..(3): Xaa - 4-nitro-phenylalanine
  MISC_FEATURE (6)..(6): Xaa - N-methylarginine
YXXLRXIR

SEQ ID NO: 28 - protein, 6 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - DArg
YXXLRX

SEQ ID NO: 29 - protein, 6 aa, Artificial Sequence
  synthetic peptide
  MISC_FEATURE (2)..(2): Xaa - Sarcosine
  MISC_FEATURE (3)..(3): Xaa - 4-chloro-phenylalanine
  MISC_FEATURE (6)..(6): Xaa - N-methylarginine
YXXLRX

SEQ ID NO: 30 - protein, 7 aa, Artificial Sequence
  synthetic peptide
  MISC_FEATURE (2)..(2): Xaa - Sarcosine
  MISC_FEATURE (3)..(3): Xaa - 4-nitro-phenylalanine
  MISC_FEATURE (6)..(6): Xaa - N-methylarginine
  MISC_FEATURE (7)..(7): Xaa - DArg
YXXLRXX

SEQ ID NO: 31 - protein, 6 aa, Artificial Sequence
  synthetic peptide
  MISC_FEATURE (2)..(2): Xaa - Sarcosine
  MISC_FEATURE (3)..(3): Xaa - 4-nitro-phenylalanine
  MISC_FEATURE (6)..(6): Xaa - DArg
YXXLRX

SEQ ID NO: 32 - protein, 4 aa, Artificial Sequence
  synthetic peptide
  MISC_FEATURE (1)..(2): Xaa - DPhe
  MISC_FEATURE (3)..(3): Xaa - DLeu
  MISC_FEATURE (4)..(4): Xaa - DLys
XXXX

SEQ ID NO: 33 - protein, 6 aa, Artificial Sequence
  synthetic peptide
FFLGRR

SEQ ID NO: 34 - protein, 17 aa, Homo sapiens
YGGFLRRIRPKLKWDNQ

SEQ ID NO: 35 - protein, 4 aa, Homo sapiens
FFLK

SEQ ID NO: 36 - protein, 6 aa, Artificial Sequence
  synthetic peptide
FFLGRR

SEQ ID NO: 37 - protein, 17 aa, Artificial Sequence
  synthetic peptide
YGGFLRRIRPKLKWDNQ

SEQ ID NO: 38 - protein, 6 aa, Artificial Sequence
  synthetic peptide
YGGLRR

SEQ ID NO: 39 - protein, 7 aa, Artificial Sequence
  synthetic peptide
YGGFLRH

SEQ ID NO: 40 - protein, 7 aa, Artificial Sequence
  synthetic peptide
YGGFLRN

SEQ ID NO: 41 - protein, 7 aa, Artificial Sequence
  synthetic peptide
  MISC_FEATURE (4)..(4): Xaa - 4-nitro-phenylalanine
YGGXLRR

SEQ ID NO: 42 - protein, 7 aa, Artificial Sequence
  synthetic peptide
  MISC_FEATURE (4)..(4): Xaa - 4-methoxy-Tyrosine
YGGXLRR

SEQ ID NO: 43 - protein, 7 aa, Artificial Sequence
  synthetic peptide
  MISC_FEATURE (4)..(4): Xaa - 2-amido-phenylanine
YGGXLRR

SEQ ID NO: 44 - protein, 7 aa, Artificial Sequence
  synthetic peptide
YGGFLRH

SEQ ID NO: 45 - protein, 7 aa, Artificial Sequence
  synthetic peptide
YGGFLRN

SEQ ID NO: 46 - protein, 6 aa, Artificial Sequence
  synthetic peptide
YGGFLR

SEQ ID NO: 47 - protein, 6 aa, Artificial Sequence
  synthetic peptide
  MISC_FEATURE (3)..(3): Xaa - DAla
YGGFLR

SEQ ID NO: 48 - protein, 6 aa, Artificial Sequence
  synthetic peptide
YGGFLK

SEQ ID NO: 49 - protein, 6 aa, Artificial Sequence
  synthetic peptide
GGFLRR

SEQ ID NO: 50 - protein, 7 aa, Artificial Sequence
  synthetic peptide
  MISC_FEATURE (6)..(6): Xaa - citruline
YGGFLXR

SEQ ID NO: 51 - protein, 7 aa, Artificial Sequence
  synthetic peptide
YGGFLNR

SEQ ID NO: 52 - protein, 7 aa, Artificial Sequence
  synthetic peptide
YGAFLRR

SEQ ID NO: 53 - protein, 7 aa, Artificial Sequence
  synthetic peptide
  MISC_FEATURE (4)..(4): Xaa - 2-thienyl alanine
YGGXLNR

SEQ ID NO: 54 - protein, 6 aa, Artificial Sequence
  synthetic peptide
  MISC_FEATURE (2)..(2): Xaa - Sarcosine
YXGFLR

SEQ ID NO: 55 - protein, 6 aa, Artificial Sequence
  synthetic peptide
YGGFLK

SEQ ID NO: 56 - protein, 7 aa, Artificial Sequence
  synthetic peptide
YGGFLRR

SEQ ID NO: 57 - protein, 7 aa, Artificial Sequence
  synthetic peptide
  MISC_FEATURE (4)..(4): Xaa - 4-nitro-phenylalanine
  MISC_FEATURE (6)..(6): Xaa - DArg
YGGXLXR

SEQ ID NO: 58 - protein, 7 aa, Artificial Sequence
  synthetic peptide
  MISC_FEATURE (2)..(2): Xaa = Sarcosine
  MISC_FEATURE (4)..(4): Xaa = 4-nitro-phenylalanine
  MISC_FEATURE (7)..(7): Xaa = DArg
YXGXLRX

SEQ ID NO: 59 - protein, 7 aa, Artificial Sequence
  synthetic peptide
  MISC_FEATURE (3)..(3): Xaa = Sarcosine
  MISC_FEATURE (4)..(4): Xaa = 4-nitro-phenylalanine
  MISC_FEATURE (7)..(7): Xaa = DArg
YGXXLRX

SEQ ID NO: 60 - protein, 7 aa, Artificial Sequence
  synthetic peptide
  MISC_FEATURE (3)..(3): Xaa = Sarcosine
  MISC_FEATURE (4)..(4): Xaa = 4-nitro-phenylalanine
  MISC_FEATURE (6)..(6): Xaa = DArg
YGXXLXR

SEQ ID NO: 61 - protein, 7 aa, Artificial Sequence
  synthetic peptide
  MISC_FEATURE (2)..(2): Xaa = Sarcosine
  MISC_FEATURE (4)..(4): Xaa = 4-nitro-phenylalanine
  MISC_FEATURE (6)..(6): Xaa = DArg
YXGXLXR

SEQ ID NO: 62 - protein, 7 aa, Artificial Sequence
  synthetic peptide
  MISC_FEATURE (4)..(4): Xaa = 4-nitro-phenylalanine
  MISC_FEATURE (6)..(6): Xaa = DArg
YGGXLXR

SEQ ID NO: 63 - protein, 7 aa, Artificial Sequence
  synthetic peptide
  MISC_FEATURE (2)..(2): Xaa = Sarcosine
  MISC_FEATURE (4)..(4): Xaa = 4-nitro-phenylalanine
  MISC_FEATURE (6)..(7): Xaa = DArg
YXGXLXX

(19) United States
(12) Patent Application Publication
(10) Pub. No.: US 2024/0301023 A1
(43) Pub. Date: Sep. 12, 2024

(54) PEPTIDES AND USES THEREOF

(71) Applicant: PreveCeutical Medical Inc., Vancouver (CA)

(72) Inventors: Harendra PAREKH, Woolloongabba, Queensland (AU); Karnaker TUPALLY, St Lucia, Queensland (AU); Peter CABOT, St Lucia, Queensland (AU); Ajit Nandakumar KANDALE, Capalaba, Queensland (AU)

(21) Appl. No.: 18/003,768

(22) PCT Filed: Jul. 1, 2021
(86) PCT No.: PCT/AU2021/050707

§ 371 Date: Dec. 29, 2022

(30) Foreign Application Priority Data
Jul. 1, 2020 (AU) 2020902233

(51) Int. Cl. C07K 14/665; A61K 38/00; A61P 25/04

(52) U.S. Cl. CPC C07K 14/665; A61P 25/04; A61K 38/00

(57) ABSTRACT

Peptide analogues of dynorphin and their use in pain management, the peptide analogues having good biological stability and reduced side effects compared to opioid analgesics.

### FIELD OF THE INVENTION

The present invention relates to peptide analogues of dynorphin and their use in pain management.

### REFERENCE TO SEQUENCE LISTING

The present application is being filed along with a Sequence Listing in electronic format. The Sequence Listing is provided as a file entitled 59420935_1, created and last modified on Apr. 18, 2024, which is 20,170 bytes in size. The information in the electronic format of the Sequence Listing is incorporated herein by reference in its entirety.

### BACKGROUND OF THE INVENTION

Opioids are a class of drugs that are used clinically as painkillers. As such, opioids are a mainstay of pain management and are considered the gold standard. However, opioids such as morphine have significant side-effects including constipation, sedation, respiratory depression, dependence and tolerance. These side-effects add significant burden to the quality of life experienced by patients, with prevention and management of opioid dependence being particularly challenging.

Opioids mainly act via the opioid receptors (μ, δ, κ and nociceptin). It is postulated that some of the undesirable side-effects reside in the agonist activity on some of these opioid receptors. As such, it would be advantageous to provide an opioid that has selective activity on some receptors to ameliorate this issue.

The natural mechanism for analgesia involves endogenous opioids. One such endogenous opioid is dynorphin which arises from prodynorphin. However, dynorphins are metabolised relatively quickly and so it would be advantageous to provide dynorphins which have greater pharmacokinetic (metabolic) stability and thus a longer half-life.

There is a need for the development of new drugs that are effective in pain management, while also having less side-effects and greater in vivo stability. There is also a need for a larger selection of pain management drugs to choose from.

### SUMMARY OF THE INVENTION

The present invention is predicated at least in part on the discovery of peptidic dynorphin analogues that have selective activity in relation to opioid receptors and have improved stability in vivo allowing longer lasting pain management.

In one aspect of the present invention there is provided a compound of formula (I), or a pharmaceutically acceptable salt, solvate, stereoisomer or prodrug thereof:

R₁NH—X₁—X₂—X₃—X₄—X₅—X₆—X₇—X₈—X₉—X₁₀—X₁₁—C(O)R₂ (I)
wherein R₁ is selected from the group consisting of hydrogen and C₁₋₆alkyl;
R₂ is selected from the group consisting of OH, NH₂, NH(C₁₋₆alkyl) and N(C₁₋₆alkyl)₂;
X₁ is selected from the group consisting of L-tyrosine, D-tyrosine, L-3-(4-pyridyl)-alanine and D-3-(4-pyridyl)-alanine, L-phenylalanine, D-phenylalanine, a tyrosine derivative, a 3-(4-pyridyl)-alanine derivative and a phenylalanine derivative;
X₂ is selected from the group consisting of glycine, sarcosine, N-alkylglycine, 4-aminobutyric acid, L-leucine, D-leucine, L-isoleucine, D-isoleucine, L-valine, D-valine, L-alanine, D-alanine, L-3-(4-pyridyl)-alanine and D-3-(4-pyridyl)-alanine;
X₃ is absent;
X₄ is selected from the group consisting of L-phenylalanine, D-phenylalanine, L-leucine, D-leucine, a phenylalanine derivative and a leucine derivative;
X₅ is selected from the group consisting of glycine, L-leucine, D-leucine, L-isoleucine, D-isoleucine, L-valine and D-valine;
X₆ is selected from the group consisting of a positively charged amino acid residue, a negatively charged amino acid residue and a polar uncharged amino acid residue;
X₇ is selected from the group consisting of a positively charged amino acid residue, a negatively charged amino acid residue and a polar uncharged amino acid residue;
X₈ is absent or is selected from the group consisting of a hydrophobic amino acid residue and —C₁₋₁₀ alkylene-;
X₉ is absent or is selected from the group consisting of a positively charged amino acid residue and a polar uncharged amino acid residue;
X₁₀ is absent or is a hydrophobic amino acid residue; and
X₁₁ is absent or is a positively charged amino acid residue;
wherein at least one amino acid residue X₁, X₂ and X₄ to X₇ is a non-proteinogenic amino acid;
or a pharmaceutically acceptable salt, solvate, stereoisomer or prodrug thereof.

In another aspect of the present invention there is provided a pharmaceutical composition comprising a compound of formula (I) or pharmaceutically acceptable salt, solvate, stereoisomer or prodrug thereof and a pharmaceutically acceptable carrier, diluent and/or excipient.

In yet another aspect of the invention, there is provided a method of treating or preventing pain in a subject comprising administering an effective amount of a compound of formula (I) or a pharmaceutically acceptable salt, solvate, stereoisomer or prodrug thereof, or a pharmaceutical composition comprising a compound of formula (I) or a pharmaceutically acceptable salt, solvate, stereoisomer or prodrug thereof.

In a further aspect of the present invention, there is provided a use of a compound of formula (I) or a pharmaceutically acceptable salt, solvate, stereoisomer of prodrug thereof in the manufacture of a medicament for treating or preventing pain.

In yet a further aspect of the present invention, there is provided a compound of formula (I) or a pharmaceutically acceptable salt thereof for use in treating or preventing pain.

### BRIEF DESCRIPTION OF THE FIGURES

FIG. 1 provides graphical representations of concentration-response curves in the cAMP Alpha Screen assay for peptides of a) SEQ ID NO 2, b) SEQ ID NO. 3, c) SEQ ID NO. 4, d) SEQ ID NO. 35 (CR845) and e) positive controls. Dotted lines represent those agonists with lower efficacy having two independent experiments, whilst solid lines are represented by a minimum of 3 independent experiments. Data shows mean±SD for solid lines only.

FIG. 2 provides graphical representations of cAMP activity tested for SEQ ID No. 2 in the presence of Naloxone, together with (SEQ ID NO.35) and Dyn1-17 (SEQ ID NO. 37) controls in KOR cells.

FIG. 3 provides a graphical representation of cAMP modulation by naloxone for SEQ ID Nos 4, 19, 20 and 24. Naloxone significantly inhibited peptide activity at KORs. Mean±SD. *p<0.05 Student's paired T-Test from same compound in absence of naloxone.

FIG. 4 provides graphical representations of KOR desensitization in cAMP assay in response to peptides of SEQ ID Nos 4, 18, 19, 20 and 24 (A) with a comparison to morphine (B). Following 6 hrs of pre-treatment with respective agonists (1 μM), KOR and MOR for morphine, cells were re-stimulated with the same peptides/compound in a concentration-response manner and cAMP production measured (circles), compared to non-pre-treated cells as control (squares). Mean±*p<0.0001 unpaired two-tailed T-test maximal response (% change from max) compared to control.

FIG. 5 is a graphical representation showing the Bias factor β, relative to U50488H. Positive values indicate cAMP modulation bias, negative values indicate a pERK bias. U50488H has a bias of 0.

FIG. 6 shows graphical representations of mechanical paw withdrawal threshold over time as measured by Randal Selitto assay in the FCA model of inflammatory pain. The graphs show the grams of force required to elicit a paw-withdrawal response for individual peptides tested in the FCA-treated paw (red) and uninflamed paw (contralateral, black) post-compound administration (after t=0). n=6/group, mean±SEM. Force required to elicit a paw withdrawal response in the contralateral/uninflamed paw at baseline (t=0, prior to compound admin.) and at t=120 following compound administration in the ipsilateral paw. Mean±SEM. * p<0.05 paired t-test.

### DESCRIPTION OF THE INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 20%, 15% or 10% to a reference quantity, level, value, dimension, size, or amount.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “amino acid” refers to an α-amino acid or a 3-amino acid and may be a L- or D-isomer. The amino acid may have a naturally occurring side chain (see Table 1) or a non-proteinogenic side chain (see Table 2). The amino acid may also be further substituted in the α-position or the β-position with a group selected from —C₁₋₆alkyl, —(CH₂)CORₐ, —(CH₂)ₙR_{b} and —PO₃H, where Rₐ is —OH, —NH₂, —NHC₁₋₃alkyl, —OC₁₋₃alkyl or —C₁₋₃alkyl and R_{b} is —OH, —SH, —SC₁₋₃alkyl, —OC₁₋₃alkyl, —NH₂, —NHC₁₋₃alkyl or —NHC(C═NH)NH₂ and where each alkyl group may be substituted with one or more groups selected from —OH, —NH₂, —NHC₁₋₃alkyl, —OC₁₋₃alkyl, —SH, —SC₁₋₃alkyl, —CO₂H, —CO₂C₁₋₃alkyl, —CONH₂ and —CONHC₁₋₃alkyl.

Amino acid structure and single and three letter abbreviations used throughout the specification are defined in Table 1, which lists the twenty naturally occurring amino acids which occur in proteins as L-isomers.

TABLE 1

| 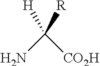 | 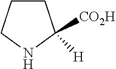 |
|---|---|
| (1) | (2) |

| Amino | Three-letter | One-letter | Structure of side |
|---|---|---|---|
| Acid | Abbreviation | symbol | chain (R) |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indolyl) |
| Tyrosine | Tyr | Y | CH₂(4-hydroxyphenyl) |
| Valine | Val | V | —CH(CH₃)₂ |

The term “non-proteinogenic amino acid” as used herein, refers to amino acids having a side chain that does not occur in the naturally occurring L-α-amino acids recited in Table 1. Examples of non-proteinogenic amino acids and derivatives include, but are not limited to, norleucine, 4-aminobutyric acid, 4-amino-3-hydroxy-5-phenylpentanoic acid, 6-aminohexanoic acid, t-butylglycine, norvaline, phenylglycine, ornithine, citrulline, sarcosine, 4-amino-3-hydroxy-6-methylheptanoic acid, 2-thienyl alanine and/or D-isomers of natural amino acids. A list of unnatural amino acids that may be useful herein is shown in Table 2.

TABLE 2

| Amino Acid | Structure |
|---|---|
| sarcosine | 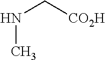 |
| 4(γ)- aminobutyric acid | 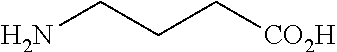 |
| citrulline | 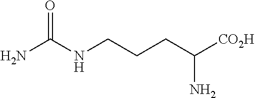 [SMILES: NC(=O)NCCCC(N)C(=O)O] |
| norvaline | 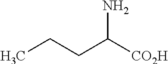 |
| ornithine | 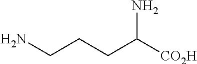 |
| phenylglycine | 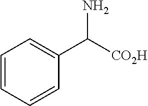 [SMILES: NC(C(=O)O)c1ccccc1] |
| 2-thienyl alanine (Tal) | 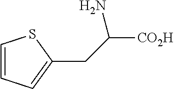 [SMILES: NC(Cc1cccs1)C(=O)O] |
| homoarginine | 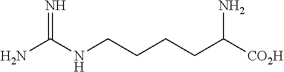 [SMILES: N=C(N)NCCCCC(N)C(=O)O] |
| diaminobutyric acid | 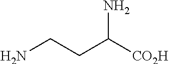 |
| homotyrosine | 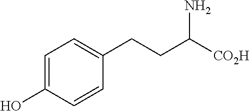 [SMILES: NC(CCc1ccc(O)cc1)C(=O)O] |
| O-methyl- tyrosine | 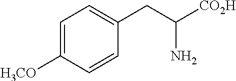 [SMILES: COc1ccc(CC(N)C(=O)O)cc1] |
| O-methyl homotyrosine | 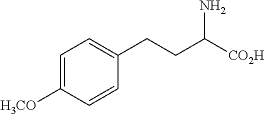 [SMILES: COc1ccc(CCC(N)C(=O)O)cc1] |
| 4-carboxy phenylalanine | 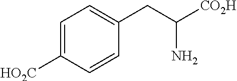 [SMILES: NC(Cc1ccc(C(=O)O)cc1)C(=O)O] |
| Cyclohexyl- alanine | 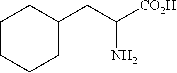 [SMILES: NC(CC1CCCCC1)C(=O)O] |
| Cyclopentyl alanine | 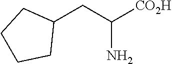 [SMILES: NC(CC1CCCC1)C(=O)O] |
| norleucine | 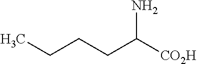 |
| 4-nitro phenylalanine | 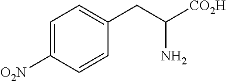 [SMILES: NC(Cc1ccc([N+](=O)[O-])cc1)C(=O)O] |
| 4-fluoro phenylalanine | 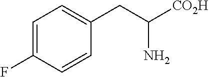 [SMILES: NC(Cc1ccc(F)cc1)C(=O)O] |
| 4-chloro phenylalanine | 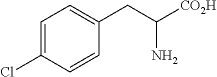 [SMILES: NC(Cc1ccc(Cl)cc1)C(=O)O] |
| 4-amino phenylalanine | 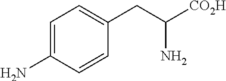 [SMILES: Nc1ccc(CC(N)C(=O)O)cc1] |
| 2-amino phenylalanine | 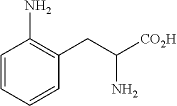 [SMILES: Nc1ccccc1CC(N)C(=O)O] |
| 2- aminomethyl- phenylalanine | 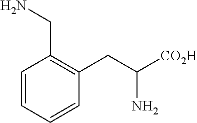 [SMILES: NCc1ccccc1CC(N)C(=O)O] |
| homoproline | 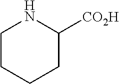 |
| 4,4-carboxy amino- piperidine | 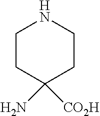 |
| 6-amino- hexanoic acid | 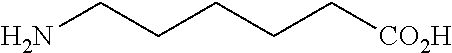 |
| 8-amino- octanoic acid | 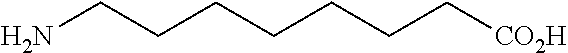 [SMILES: NCCCCCCCC(=O)O] |
| 3-(3-pyridyl)- alanine | 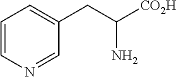 [SMILES: NC(Cc1cccnc1)C(=O)O] |
| 3-(4-pyridyl)- alanine | 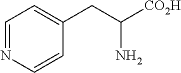 [SMILES: NC(Cc1ccncc1)C(=O)O] |
| 4-amino- benzoic acid | 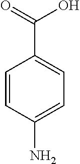 |

The non-proteinogenic amino acids in Table 2 may be in the L or D configuration and may be N-methylated on the α-amino group.

The term “alkyl” as used herein refers to straight chain or branched hydrocarbon groups, for example, alkyl groups may have 1 to 20 carbon atoms, such as 1 to 10 carbon atoms. Suitable alkyl groups include, but are not limited to methyl, ethyl, propyl, isopropyl, butyl, isobutyl, pentyl and hexyl. The term alkyl may be prefixed by a specified number of carbon atoms to indicate the number of carbon atoms or a range of numbers of carbon atoms that may be present in the alkyl group. For example, C₁₋₃alkyl refers to methyl, ethyl, propyl and isopropyl.

As used herein, the term “alkylene” refers to a divalent saturated hydrocarbon chain having 1 to 10 carbon atoms. Where appropriate, the alkylene group may have a specified number of carbon atoms, for example, C₁₋₁₀alkylene includes alkylene groups having 1, 2, 3, 4, 5, 6, 7, 8, 9 and 10 carbon atoms in a linear arrangement. Examples of suitable alkylene groups include, but are not limited to, —CH₂—, —CH₂CH₂—, —CH₂CH₂CH₂—, —CH₂CH₂CH₂CH₂—, —CH₂CH₂CH₂CH₂CH₂— and —CH₂CH₂CH₂CH₂CH₂CH₂—.

As used herein, the term “cycloalkyl” refers to a saturated cyclic hydrocarbon. The cycloalkyl ring may include a specified number of carbon atoms. For example, a 3 to 8 membered cycloalkyl group includes 3, 4, 5, 6, 7 or 8 carbon atoms. Examples of suitable cycloalkyl groups include, but are not limited to, cyclopropyl, cyclobutyl, cyclopentyl, cyclohexyl, cycloheptyl and cyclooctyl.

The term “halo” as used herein refers to fluoro, chloro, bromo and iodo.

The term “hydrophilic amino acid residue” as used herein refers to an amino acid residue in which the side chain is polar or charged. Examples include glycine, sarcosine (N-methylglycine), L-serine, L-threonine, L-cysteine, L-tyrosine, L-asparagine, L-glutamine, L-aspartic acid, L-glutamic acid, L-lysine, L-arginine, L-histidine, L-ornithine, D-serine, D-threonine, D-cysteine, D-tyrosine, D-asparagine, D-glutamine, D-aspartic acid, D-glutamic acid, D-lysine, D-arginine, D-histidine, D-ornithine, N-methylglycine, N-methyl-L-serine, N-methyl-L-threonine, N-methyl-L-cysteine, N-methyl-L-tyrosine, N-methyl-L-asparagine, N-methyl-L-glutamine, N-methyl-L-aspartic acid, N-methyl-L-glutamic acid, N-methyl-L-lysine, N-methyl-L-arginine, N-methyl-L-histidine, N-methyl-L-ornithine, N-methyl-D-serine, N-methyl-D-threonine, N-methyl-D-cysteine, N-methyl-D-tyrosine, N-methyl-D-asparagine, N-methyl-D-glutamine, N-methyl-D-aspartic acid, N-methyl-D-glutamic acid, N-methyl-D-lysine, N-methyl-D-arginine, N-methyl-D-histidine and N-methyl-D-ornithine, L-diaminobutyric acid, D-diaminobutyric acid, N-methyl-L-diaminobutyric acid, N-methyl-D-diaminobutyric acid, L-citrulline, D-citrulline, N-methyl-L-citrulline, N-methyl-D-citrulline, L-homoarginine, D-homoarginine, N-methyl-L-homoarginine, N-methyl-D-homoarginine, L-O-methyltyrosine, D-O-methyltyrosine, N-methyl-L-O-methyltyrosine, N-methyl-D-O-methyltyrosine, L-homotyrosine, D-homotyrosine, N-methyl-L-homotyrosine, N-methyl-D-homotyrosine, L-O-methylhomotyrosine, D-O-methylhomotyrosine, N-methyl-L-O-methylhomotyrosine, N-methyl-D-O-methylhomotyrosine, L-4-carboxyphenylalanine, D-4-carboxyphenylalanine, L-N-methyl-4-carboxyphenylalanine and D-N-methyl-4-carboxyphenylalanine.

As used herein, the term “hydrophobic amino acid residue” refers to an amino acid residue in which the side chain is non-polar. Examples include, but are not limited to L-alanine, L-valine, L-leucine, L-isoleucine, L-proline, L-methionine, L-phenylalanine, L-tryptophan, L-γ-aminoisobutyric acid, D-alanine, D-valine, D-leucine, D-isoleucine, D-proline, D-methionine, D-phenylalanine, D-tryptophan, D-γ-aminoisobutyric acid, L-cyclohexylalanine, D-cyclohexylalanine, L-cyclopentylalanine, D-cyclopentylalanine, L-norleucine, D-norleucine, L-norvaline, D-norvaline, L-tert-butylglycine, D-tert-butylglycine, L-ethylglycine, D-ethylglycine, 6-aminohexanoic acid, 8-aminooctanoic acid, N-methyl-L-alanine, N-methyl-L-valine, N-methyl-L-leucine, N-methyl-L-isoleucine, N-methyl-L-proline, N-methyl-L-methionine, N-methyl-L-phenylalanine, N-methyl-L-tryptophan, N-methyl-L-γ-aminoisobutyric acid, N-methyl-D-alanine, N-methyl-D-valine, N-methyl-D-leucine, N-methyl-D-isoleucine, N-methyl-D-proline, N-methyl-D-methionine, N-methyl-D-phenylalanine, N-methyl-D-tryptophan, N-methyl-D-γ-aminoisobutyric acid, N-methyl-L-cyclohexylalanine, N-methyl-D-cyclohexylalanine, N-methyl-L-cyclopentylalanine, N-methyl-D-cyclopentylalanine, N-methyl-L-norleucine, N-methyl-D-norleucine, N-methyl-L-norvaline, N-methyl-D-norvaline, N-methyl-L-tert-butylglycine, N-methyl-D-tert-butylglycine, N-methyl-L-ethylglycine, N-methyl-D-ethylglycine, L-4-nitrophenylalanine, D-4-nitrophenylalanine, L-N-methyl-4-nitrophenylalanine, D-N-methyl-4-nitrophenylalanine, L-4-halophenylalanine, D-4-halophenylalanine, L-N-methyl-4-halophenylalanine, D-N-methyl-4-halophenylalanine, L-4-aminophenylalanine, D-4-aminophenylalanine, L-N-methyl-4-aminophenylalanine, D-N-methyl-4-aminophenylalanine, L-2-aminophenylalanine, D-2-aminophenylalanine, L-N-methyl-2-aminophenylalanine, D-N-methyl-2-aminophenylalanine, L-2-aminomethylphenylalanine, D-2-aminomethylphenylalanine, L-N-methyl-2-aminomethylphenylalanine, D-N-methyl-2-aminomethylphenylalanine, L-3-(4-pyridyl)alanine, D-3-(4-pyridyl)-alanine, L-N-methyl-3-(4-pyridyl)alanine, D-N-methyl-3-(4-pyridyl)alanine, L-3-(3-pyridyl)-alanine, D-3-(3-pyridyl)-alanine, L-N-methyl-3-(3-pyridyl)-alanine, D-N-methyl-3-(3-pyridyl)alanine, 4-aminobenzoic acid, L-2-thienylalanine, D-2-thienylalanine, L-N-methyl-2-thienylalanine, D-N-methyl-2-thienylalanine, L-phenylglycine, D-phenylglycine, L-N-methyl-phenylglycine, D-N-methyl-phenylglycine, L-homoproline, D-homoproline, N-methyl-L-homoproline and N-methyl-L-homoproline.

As used herein, the term “positively charged amino acid residue” refers to an amino acid residue having a side chain capable of bearing a positive charge. Examples include, but are not limited to L-lysine, L-arginine, L-histidine, L-ornithine, D-lysine, D-arginine, D-histidine, D-ornithine, N-methyl-L-lysine, N-methyl-L-arginine, N-methyl-L-histidine, N-methyl-L-ornithine, N-methyl-D-lysine, N-methyl-D-arginine, N-methyl-D-histidine, N-methyl-D-ornithine, L-diaminobutyric acid (DAB), D-diaminobutyric acid, N-methyl-L-diaminobutyric acid, N-methyl-D-diaminobutyric acid, L-citrulline (CIT), D-citrulline, N-methyl-L-citrulline, N-methyl-D-citrulline, L-4,4-carboxyaminopiperidine (CAP), D-4,4-carboxyaminopiperidine, L-N-Methyl-4,4-carboxyaminopiperidine, D-N-Methyl-4,4-carboxyaminopiperidine, L-homoarginine, D-homoarginine, N-methyl-L-homoarginine and N-methyl-D-homoarginine.

As used herein, the term “negatively charged amino acid residue” refers to an amino acid residue having a side chain capable of bearing a negative charge. Examples include, but are not limited to L-aspartic acid, L-glutamic acid, D-aspartic acid, D-glutamic acid, N-methyl-L-aspartic acid, N-methyl-L-glutamic acid, N-methyl-D-aspartic acid, N-methyl-D-glutamic acid, L-4-carboxyphenylalanine, D-4-carboxyphenylalanine, L-N-Methyl-4-carboxyphenylalanine and D-N-Methyl-4-carboxyphenylalanine.

As used herein, the term “polar uncharged amino acid residue” refers to an amino acid residue having a side chain that is uncharged and has a dipole moment. Examples of polar amino acid residues, include, but are not limited to glycine, sarcosine, L-serine, L-threonine, L-cysteine, L-tyrosine, L-asparagine, L-glutamine, D-serine, D-threonine, D-cysteine, D-tyrosine, D-asparagine and D-glutamine, N-methyl-L-serine, N-methyl-L-threonine, N-methyl-L-cysteine, N-methyl-L-tyrosine, N-methyl-L-asparagine, N-methyl-L-glutamine, N-methyl-D-serine, N-methyl-D-threonine, N-methyl-D-cysteine, N-methyl-D-tyrosine, N-methyl-D-asparagine, N-methyl-D-glutamine, L-homoarginine, D-homoarginine, N-methyl-L-homoarginine, N-methyl-D-homoarginine, L-O-methyltyrosine, D-O-methyltyrosine, N-methyl-L-O-methyltyrosine, N-methyl-D-O-methyltyrosine, L-homotyrosine, D-homotyrosine, N-methyl-L-homotyrosine, N-methyl-D-homotyrosine, L-O-methylhomotyrosine, D-O-methylhomotyrosine, N-methyl-L-O-methylhomotyrosine and N-methyl-D-O-methylhomotyrosine.

The term “amino acid having a small side chain” refers to amino acid residues having a side chain with 4 or less non-hydrogen atoms, especially 3 or less non-hydrogen atoms. Examples include, but are not limited to, glycine, sarcosine, L-alanine, L-valine, L-leucine, L-isoleucine, L-methionine, L-serine, L-threonine, L-cysteine, L-asparagine, L-aspartic acid, D-alanine, D-valine, D-leucine, D-isoleucine, D-methionine, D-serine, D-threonine, D-cysteine, D-asparagine and D-aspartic acid, especially glycine, L-alanine, L-valine, L-serine, L-threonine, L-cysteine, L-alanine, L-valine, L-serine, L-threonine and L-cysteine.

The term “conservative amino acid substitution” refers to substituting one amino acid in a sequence with another amino acid that has similar properties of size, polarity and/or aromaticity and does not change the nature or activity of the peptide. For example, one polar amino acid residue may be substituted with another polar amino acid residue or an amino acid residue having a small side chain may be substituted with another amino acid residue having a small side chain.

The compounds of the invention may be in the form of pharmaceutically acceptable salts. It will be appreciated however that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicylic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quaternised with such agents as lower alkyl halide, such as methyl, ethyl, propyl, and butyl chlorides, bromides and iodides; dialkyl sulfates like dimethyl and diethyl sulfate; and others.

It will also be recognised that compounds of the invention may possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution. The compounds of the invention may exist as geometric isomers. The invention also relates to compounds in substantially pure cis (Z) or trans (E) or mixtures thereof.

The compounds of the invention may also be in the form of solvates, including hydrates. The term “solvate” is used herein to refer to a complex of variable stoichiometry formed by a solute (a compound of formula (I)) and a solvent. Such solvents should not interfere with the biological activity of the solute. Solvents that may be included in a solvate include, but are not limited to, water, ethanol, propanol, and acetic acid. Methods of solvation are generally known within the art.

The term “pro-drug” is used in its broadest sense and encompasses those derivatives that are converted in vivo to the compounds of formula (I). Such derivatives would readily occur to those skilled in the art and include, for example, compounds where a free hydroxy group is converted into an ester derivative or a free nitrogen is converted to an N-oxide. Examples of ester derivatives include alkyl esters, phosphate esters and those formed from amino acids. Conventional procedures for the preparation of suitable prodrugs are described in text books such as “Design of Prodrugs” Ed. H. Bundgaard, Elsevier, 1985.

### Compounds of the Invention

In one aspect of the present invention there is provided a compound of formula (I), or a pharmaceutically acceptable salt, solvate, stereoisomer or prodrug thereof:

R₁NH—X₁—X₂—X₃—X₄—X₅—X₆—X₇—X₈—X₉—X₁₀—X₁₁—C(O)R₂ (I)
wherein R₁ is selected from the group consisting of hydrogen and C₁₋₆alkyl;
R₂ is selected from the group consisting of OH, NH₂, NH(C₁₋₆alkyl) and N(C₁₋₆alkyl)₂;
X₁ is selected from the group consisting of L-tyrosine, D-tyrosine, L-phenylalanine, D-phenylalanine, L-3-(4-pyridyl)-alanine, D-3-(4-pyridyl)-alanine, a tyrosine derivative, a phenylalanine derivative and 3-(4-pyridyl)-alanine derivative;
X₂ is selected from the group consisting of glycine, sarcosine, N-alkylglycine, 4-aminobutyric acid, L-leucine, D-leucine, L-isoleucine, D-isoleucine, L-valine, D-valine, L-alanine, D-alanine, L-3-(4-pyridyl)-alanine and D-3-(4-pyridyl)-alanine;
X₃ is absent;
X₄ is selected from the group consisting of L-phenylalanine, D-phenylalanine, L-leucine or D-leucine, a phenylalanine derivative and a leucine derivative;
X₅ is selected from the group consisting of glycine, L-leucine, D-leucine, L-isoleucine, D-isoleucine, L-valine and D-valine;
X₆ is selected from the group consisting of a positively charged amino acid residue, a negatively charged amino acid residue and a polar uncharged amino acid residue;
X₇ is selected from the group consisting of a positively charged amino acid residue, a negatively charged amino acid residue and a polar uncharged amino acid residue;
X₈ is absent or is selected from the group consisting of a hydrophobic amino acid residue and —C₁₋₁₀alkylene-;
X₉ is absent or is selected from the group consisting of a positively charged amino acid residue and a polar uncharged amino acid residue;
X₁₀ is absent or is a hydrophobic amino acid residue; and
X₁₁ is absent or a positively charged amino acid residue;
wherein at least one amino acid residue X₁, X₂ and X₄ to X₇ is a non-proteinogenic amino acid, or a pharmaceutically acceptable salt, solvate, stereoisomer or prodrug thereof.

The term “tyrosine derivative” refers to D- or L-tyrosine that has further substitution on any of the carbon atoms of the phenyl ring, the methylene group attached to the alpha carbon, the phenoxy oxygen atom or the amino nitrogen. A tyrosine derivative includes the moiety:

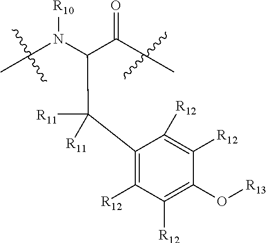

wherein R₁₀ is selected from the group consisting of hydrogen and alkyl, especially wherein the alkyl is C₁₋₆ alkyl, more especially wherein the alkyl is methyl or ethyl. In one embodiment R₁₀ is hydrogen or methyl, especially hydrogen;
wherein each R₁₁ is independently selected from the group consisting of hydrogen; alkyl, especially wherein the alkyl is C₁₋₆ alkyl, more especially wherein the alkyl is methyl or ethyl; and halo, especially wherein the halo is fluoro or chloro. In one embodiment, each R₁₁ is hydrogen;
wherein each R₁₂ is independently selected from the group consisting of hydrogen; alkyl, especially wherein the alkyl is C₁₋₆ alkyl, more especially wherein the alkyl is methyl or ethyl; halo, especially wherein the halo is fluoro or chloro; nitro; —OH and —O-alkyl, especially wherein the —O-alkyl is —O—C₁₋₆ alkyl, more especially wherein the —O-alkyl is —O—CH₃ or —O—CH₂—CH₃. In one embodiment each R₁₂ is independently selected from the group consisting of hydrogen; alkyl, especially wherein the alkyl is C₁₋₆ alkyl, more especially wherein the alkyl is methyl or ethyl; and halo, especially wherein the halo is fluoro or chloro; and

wherein R₁₃ is selected from the group consisting of hydrogen and alkyl, especially wherein the alkyl is C₁₋₆ alkyl, more especially wherein the alkyl is methyl or ethyl. In one embodiment R₁₃ is hydrogen.

The term “3-(4-pyridyl)-alanine derivative” refers to D- or L-3-(4-pyridyl)-alanine that has further substitution on any of the carbon atoms of the phenyl ring, the methylene group attached to the alpha carbon, or the amino nitrogen. A 3-(4-pyridyl)-alanine derivative includes the moiety:

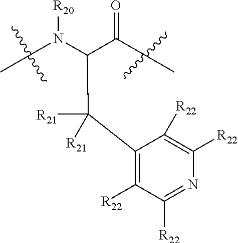

wherein R₂₀ is group consisting of hydrogen and alkyl, especially wherein the alkyl is C₁₋₆ alkyl, more especially wherein the alkyl is methyl or ethyl. In one embodiment R₂₀ is hydrogen or methyl, especially hydrogen;
wherein each R₂₁ is independently selected from the group consisting of hydrogen; alkyl, especially wherein the alkyl is C₁₋₆ alkyl, more especially wherein the alkyl is methyl or ethyl, and halo, especially wherein the halo is fluoro or chloro. In one embodiment, each R₂₁ is hydrogen; and
wherein each R₂₂ is independently selected from the group consisting of hydrogen; alkyl, especially wherein the alkyl is C₁₋₆ alkyl, more especially wherein the alkyl is methyl or ethyl; halo, especially wherein the halo is fluoro or chloro; nitro; —OH and —O-alkyl, especially wherein the —O-alkyl is —O—C₁₋₆ alkyl, more especially wherein the —O-alkyl is —O—CH₃ or —O—CH₂—CH₃. In one embodiment each R₂₂ is independently selected from the group consisting of hydrogen; alkyl, especially wherein the alkyl is C₁₋₆ alkyl, more especially wherein the alkyl is methyl or ethyl; and halo, especially wherein the halo is fluoro or chloro.

The term “phenylalanine derivative” refers to D- or L-phenylalanine that has further substitution on any of the carbon atoms of the phenyl ring, the methylene group attached to the alpha carbon, or the amino nitrogen. A phenylalanine derivative includes the moiety:

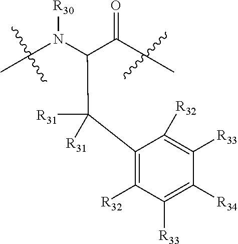

wherein R₃₀ is selected from the group consisting of hydrogen and alkyl, especially wherein the alkyl is C₁₋₆ alkyl, more especially wherein the alkyl is methyl or ethyl. In one embodiment R₃₀ is hydrogen or methyl, especially hydrogen;
wherein each R₃₁ is independently selected from the group consisting of hydrogen; alkyl, especially wherein the alkyl is C₁₋₆ alkyl, more especially wherein the alkyl is methyl or ethyl; and halo, especially wherein the halo is fluoro or chloro. In one embodiment, each R₃₁ is hydrogen;
wherein each R₃₂ is independently selected from the group consisting of hydrogen; alkyl, especially wherein the alkyl is C₁₋₆ alkyl, more especially wherein the alkyl is methyl or ethyl; halo, especially wherein the halo is fluoro or chloro; nitro; NH₂; NH(C₁₋₆alkyl); N(C₁₋₆alkyl)₂; CH₂NH₂; CH₂NH(C₁₋₆alkyl); CH₂N(C₁₋₆alkyl)₂; —OH and —O-alkyl, especially wherein the —O-alkyl is —O—C₁₋₆ alkyl, more especially wherein the —O-alkyl is —O—CH₃ or —O—CH₂—CH₃. In one embodiment each R₃₂ is independently selected from the group consisting of hydrogen; alkyl, especially wherein the alkyl is C₁₋₆ alkyl, more especially wherein the alkyl is methyl or ethyl; NH₂; CH₂NH₂ and halo, especially wherein the halo is fluoro or chloro;
wherein each R₃₃ is independently selected from the group consisting of hydrogen; alkyl, especially wherein the alkyl is C₁₋₆ alkyl, more especially wherein the alkyl is methyl or ethyl; halo, especially wherein the halo is fluoro or chloro; nitro; NH₂; NH(C₁₋₆alkyl); N(C₁₋₆alkyl)₂; CH₂NH₂; CH₂NH(C₁₋₆alkyl); CH₂N(C₁₋₆alkyl)₂; —OH and —O-alkyl, especially wherein the —O-alkyl is —O—C₁₋₆ alkyl, more especially wherein the —O-alkyl is —O—CH₃ or —O—CH₂—CH₃. In one embodiment each R₃₃ is independently selected from the group consisting of hydrogen; alkyl, especially wherein the alkyl is C₁₋₆ alkyl, more especially wherein the alkyl is methyl or ethyl; and halo, especially wherein the halo is fluoro or chloro; and
wherein R₃₄ is selected from the group consisting of hydrogen; alkyl, especially wherein the alkyl is C₁₋₆ alkyl, more especially wherein the alkyl is methyl or ethyl; halo, especially wherein the halo is fluoro or chloro; nitro; NH₂; NH(C₁₋₆alkyl); N(C₁₋₆alkyl)₂; CH₂NH₂; CH₂NH(C₁₋₆alkyl); CH₂N(C₁₋₆alkyl)₂; and —O-alkyl, especially wherein the —O-alkyl is —O—C₁₋₆ alkyl, more especially wherein the —O-alkyl is —O—CH₃ or —O—CH₂—CH₃. In one embodiment R₃₄ is selected from the group consisting of hydrogen, halo and nitro, especially hydrogen, nitro, fluoro or chloro.

The term “leucine derivative” refers to D- or L-phenylalanine that has further substitution on any of the carbon atoms or the amino nitrogen. A leucine derivative includes the moiety:

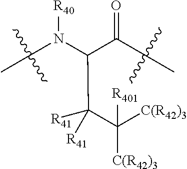

wherein R₄₀ is selected from the group consisting of hydrogen and alkyl, especially wherein the alkyl is C₁₋₆ alkyl, more especially wherein the alkyl is methyl or ethyl. In one embodiment R₄₀ is hydrogen or methyl, especially hydrogen;
wherein each R₄₁ is independently selected from the group consisting of hydrogen; halo, especially wherein the halo is fluoro or chloro; and cycloalkyl, especially wherein the cycloalkyl group is cyclopentyl, cyclohexyl or cycloheptyl. In one embodiment each R₄₁ is hydrogen or halo, especially hydrogen, fluoro or chloro; more especially each R₄₁ is hydrogen; and
wherein each R₄₂ is independently selected from the group consisting of hydrogen; halo, especially wherein the halo is fluoro or chloro; and cycloalkyl, especially wherein the cycloalkyl is cyclopentyl, cyclohexyl or cycloheptyl. In one embodiment each R₄₂ is hydrogen or halo, especially hydrogen, fluoro or chloro; more especially each R₄₂ is hydrogen.

In some embodiments, the amino acid residues are all in the L-configuration. In other embodiments, one or more amino acid residues are in the D-configuration. For example, one of X₁, X₂ and X₄ to X₇ is in the D-configuration, two of X₁, X₂ and X₄ to X₇ are in the D-configuration, three of X₁, X₂ and X₄ to X₇ are in the D-configuration, four of X₁, X₂ and X₄ to X₇ are in the D-configuration, 5 of X₁, X₂ and X₄ to X₇ are in the D-configuration, six of X₁, X₂ and X₄ to X₇ are in the D-configuration or all of X₁, X₂ and X₄ to X₇ are in the D-configuration. In particular embodiments, none, one, two or three of X₁, X₂ and X₄ to X₇ are in the D-configuration, especially, none, one or two of X₁, X₂ and X₄ to X₇ are in the D-configuration.

In some embodiments, one or both of X₆ and X₇ are in the D-configuration.

In some embodiments, X₄ is a non-natural amino acid residue. In particular embodiments, X₄ is a phenylalanine derivative. Suitable phenylalanine derivatives include 4-nitrophenylalanine, 4-chlorophenylalanine and 4-fluorophenylalanine.

In some embodiments, R₁ is hydrogen. In other embodiments, R₁ is methyl.

In some embodiments, R₂ is OH. In other embodiments, R₂ is NH₂.

In some embodiments, one or more of the following applies:
i) R₁ is hydrogen or C₁₋₃alkyl, especially hydrogen or CH₃, more especially hydrogen.
ii) R₂ is OH, NH₂, NH(C₁₋₃alkyl) or N(C₁₋₃alkyl)₂, especially OH, NH₂, NH(CH₃) or N(CH₃)₂, more especially OH and NH₂.
iii) X₁ is L-tyrosine, phenylalanine, L-N-methyltyrosine or L-3-(4-pyridyl)-alanine, especially L-tyrosine.
iv) X₂ is glycine, sarcosine, γ-aminobutyric acid, L-alanine, or L-3-(4-pyridyl)-alanine, especially glycine or sarcosine.
v) X₄ is L-phenylalanine, D-phenylalanine or a phenylalanine derivative selected from L-4-nitrophenylalanine, L-4-chlorophenylalanine, L-4-fluorophenylalanine, D-4-nitrophenylalanine, D-4-chlorophenylalanine and D-4-fluorophenylalanine especially L-phenylalanine or a phenylalanine derivative selected from L-4-nitrophenylalanine, L-4-chlorophenylalanine and L-4-fluorophenylalanine.
vi) X₅ is L-leucine;
vii) X₆ is L-lysine, L-arginine, L-histidine, L-ornithine, D-lysine, D-arginine, D-histidine, D-ornithine, N-methyl-L-lysine, N-methyl-L-arginine, N-methyl-L-histidine, N-methyl-L-ornithine, N-methyl-D-lysine, N-methyl-D-arginine, N-methyl-D-histidine, N-methyl-D-ornithine, L-diaminobutyric acid, D-diaminobutyric acid, N-methyl-L-diaminobutyric acid, N-methyl-D-diaminobutyric acid, L-citrulline, D-citrulline, N-methyl-L-citrulline, N-methyl-D-citrulline, L-homoarginine, D-homoarginine, N-methyl-L-homoarginine, N-methyl-D-homoarginine, L-asparagine, L-glutamine, D-asparagine, D-glutamine, N-methyl-L-asparagine, N-methyl-L-glutamine, N-methyl-D-asparagine or N-methyl-D-glutamine, especially L-arginine, L-histidine, L-lysine, D-arginine, D-histidine, D-lysine, N-methyl-L-arginine, N-methyl-D-arginine, L-diaminobutyric acid, D-diaminobutyric acid, L-citrulline, D-citrulline, N-methyl-L-citrulline, N-methyl-D-citrulline, L-asparagine, D-asparagine, N-methyl-L-asparagine or N-methyl-D-asparagine, more especially L-arginine, L-histidine, L-lysine, D-arginine, N-methyl-L-arginine, L-diaminobutyric acid, L-citrulline or L-asparagine, even more especially L-arginine, L-lysine, D-arginine and N-methyl-L-arginine.
viii) X₇ is selected from L-lysine, L-arginine, L-histidine, L-ornithine, D-lysine, D-arginine, D-histidine, D-ornithine, N-methyl-L-lysine, N-methyl-L-arginine, N-methyl-L-histidine, N-methyl-L-ornithine, N-methyl-D-lysine, N-methyl-D-arginine, N-methyl-D-histidine, N-methyl-D-ornithine, L-diaminobutyric acid, D-diaminobutyric acid, N-methyl-L-diaminobutyric acid, N-methyl-D-diaminobutyric acid, L-citrulline, D-citrulline, N-methyl-L-citrulline, N-methyl-D-citrulline, L-homoarginine, D-homoarginine, N-methyl-L-homoarginine, N-methyl-D-homoarginine, L-4,4-carboxyaminopiperidine, D-4,4-carboxyaminopiperidine, L-N-Methyl-4,4-carboxyaminopiperidine, D-N-Methyl-4,4-carboxyaminopiperidine, L-asparagine, L-glutamine, D-asparagine, D-glutamine, N-methyl-L-asparagine, N-methyl-L-glutamine, N-methyl-D-asparagine or N-methyl-D-glutamine, especially L-arginine, L-histidine, L-lysine, D-arginine, D-histidine, D-lysine, N-methyl-L-arginine, N-methyl-D-arginine, L-diaminobutyric acid, D-diaminobutyric acid, L-homoarginine, L-4,4-carboxyaminopiperidine, L-asparagine or D-asparagine, more especially L-arginine, L-histidine, L-lysine, D-arginine, D-histidine, D-lysine, N-methyl-L-arginine, L-diaminobutyric acid, L-homoarginine, L-4,4-carboxyaminopiperidine, or L-asparagine, even more especially L-arginine, L-lysine, D-arginine and N-methyl-L-arginine.
ix) X₈ is absent or is —C₄₋₈alkylene-, L-alanine, L-valine, L-leucine, L-isoleucine, L-proline, L-methionine, L-phenylalanine, L-tryptophan, L-γ-aminoisobutyric acid, D-alanine, D-valine, D-leucine, D-isoleucine, D-proline, D-methionine, D-phenylalanine, D-tryptophan, D-γ-aminoisobutyric acid, L-cyclohexylalanine, D-cyclohexylalanine, L-cyclopentylalanine, D-cyclopentylalanine, L-norleucine, D-norleucine, L-norvaline, D-norvaline, L-tert-butylglycine, D-tert-butylglycine, L-ethylglycine, D-ethylglycine, N-methyl-L-alanine, N-methyl-L-valine, N-methyl-L-leucine, N-methyl-L-isoleucine, N-methyl-L-proline, N-methyl-L-methionine, N-methyl-L-phenylalanine, N-methyl-L-tryptophan, N-methyl-L-γ-aminoisobutyric acid, N-methyl-D-alanine, N-methyl-D-valine, N-methyl-D-leucine, N-methyl-D-isoleucine, N-methyl-D-proline, N-methyl-D-methionine, N-methyl-D-phenylalanine, N-methyl-D-tryptophan, N-methyl-D-γ-aminoisobutyric acid, N-methyl-L-cyclohexylalanine, N-methyl-D-cyclohexylalanine, N-methyl-L-cyclopentylalanine, N-methyl-D-cyclopentylalanine, N-methyl-L-norleucine, N-methyl-D-norleucine, N-methyl-L-norvaline, N-methyl-D-norvaline, N-methyl-L-tert-butylglycine, N-methyl-D-tert-butylglycine, N-methyl-L-ethylglycine, N-methyl-D-ethylglycine, L-4-nitrophenylalanine, D-4-nitrophenylalanine, L-N-methyl-4-nitrophenylalanine, D-N-methyl-4-nitrophenylalanine, L-4-halophenylalanine, D-4-halophenylalanine, L-N-methyl-4-halophenylalanine, D-N-methyl-4-halophenylalanine, L-4-aminophenylalanine, D-4-aminophenylalanine, L-N-methyl-4-aminophenylalanine, D-N-methyl-4-aminophenylalanine, L-2-aminophenylalanine, D-2-aminophenylalanine, L-N-methyl-2-aminophenylalanine, D-N-methyl-2-aminophenylalanine, L-2-aminomethylphenylalanine, D-2-aminomethylphenylalanine, L-N-methyl-2-aminomethylphenylalanine, D-N-methyl-2-aminomethylphenylalanine, L-3-(4-pyridyl)-alanine, D-3-(4-pyridyl)-alanine, L-N-methyl-3-(4-pyridyl)-alanine, D-N-methyl-3-(4-pyridyl)-alanine, L-2-thienylalanine, D-2-thienylalanine, L-N-methyl-2-thienylalanine, D-N-methyl-2-thienylalanine, L-phenylglycine, D-phenylglycine, L-N-methyl-phenylglycine, D-N-methyl-phenylglycine, L-homoproline, D-homoproline, N-methyl-L-homoproline or N-methyl-L-homoproline, especially where X₈ is absent or is —C₅₋₇alkylene-L-alanine, L-valine, L-leucine, L-isoleucine, L-methionine, D-alanine, D-valine, D-leucine, D-isoleucine or D-methionine; more especially where X₈ is L-isoleucine or —C₅₋₇alkylene-.
x) X₉ is absent or is L-lysine, L-arginine, L-histidine, L-ornithine, D-lysine, D-arginine, D-histidine, D-ornithine, L-diaminobutyric acid, D-diaminobutyric acid, L-citrulline, D-citrulline, L-homoarginine or D-homoarginine, especially where X₉ is absent or is L-arginine or D-arginine.
xi) X₁₀ is absent or is L-proline, D-proline, L-alanine, L-valine, L-leucine, L-isoleucine, L-methionine, D-alanine, D-valine, D-leucine, D-isoleucine or D-methionine, especially where X₁₀ is absent or is L-proline.
xii) X₁₁ is absent or is L-lysine, L-arginine, L-histidine, L-ornithine, D-lysine, D-arginine, D-histidine, D-ornithine, L-diaminobutyric acid, D-diaminobutyric acid, L-citrulline, D-citrulline, L-homoarginine or D-homoarginine, especially where X₁₁ is absent or is L-lysine.

In some particular embodiments, X₄ is a substituted phenylalanine. In some embodiments, one or both of X₆ and X₇ is selected from D-arginine and N-methyl-L-arginine. In some embodiments, X₄ is a substituted phenylalanine and one or both of X₆ or X₇ is selected from D-arginine and N-methyl-L-arginine.

Particular embodiments of the compounds of formula (I) include:

| SEQ | |
|---|---|
| ID | |
| NO. | Sequence |
| 1 | NH₂-Tyr-Gaba-Phe-Leu-Arg-Arg-NH₂ |
| 2 | NH₂-Tyr-Sar-Phe-Leu-Arg-Arg-NH₂ |
| 3 | NH₂-Tyr-Sar-Phe-Leu-Arg-DArg-NH₂ |
| 4 | NH₂-Tyr-Sar-(4-NO₂)Phe-Leu-Arg-DArg-NH₂ |
| 5 | NH₂-Tyr-Sar-(4-NO₂)Phe-Leu-DArg-Arg-NH₂ |
| 6 | NH₂-Tyr-Sar-Phe-Leu-DArg-Arg-NH₂ |
| 7 | NH₂-Tyr-Sar-Phe-Leu-DArg-DArg-NH₂ |
| 8 | NH₂-Tyr- Gly-(4-NO₂)Phe-Leu-DArg-DArg-NH₂ |
| 9 | NH₂-Phe-Sar-(4-NO₂)Phe-Leu-DArg-DArg-NH₂ |
| 10 | NH₂-Tyr-Sar-(4-NO₂)Phe-Leu-DLys-DArg-NH₂ |
| 11 | NH₂-Tyr-Sar-(4-Cl)Phe-Leu-Arg-DArg-NH₂ |
| 12 | NH₂-Tyr-Sar-(4-F)Phe-Leu-Arg-DArg-NH₂ |
| 13 | NH₂-Tyr-Sar-(4-Cl)Phe-Leu-NMA-DArg-NH₂ |
| 14 | NH₂-Tyr-Sar-(4-F)Phe-Leu-NMA-DArg-NH₂ |
| 15 | NH₂-Tyr-Sar-(4-NO₂)Phe-Leu-NMA-DArg-NH₂ |
| 16 | NH₂-Tyr-Sar-(4-Cl)Phe-Leu-Lys-DArg-NH₂ |
| 17 | NH₂-Tyr-Sar-(4-Cl)Phe-Leu-Arg-NMA-NH₂ |
| 18 | NH₂-Tyr-Sar-(4-NO₂)Phe-Leu-Arg-NMA-NH₂ |
| 19 | NH₂-Tyr-Sar-(4-NO₂)Phe-Leu-NMA-NMA-NH₂ |
| 20 | NH₂-Tyr-Sar-(4-Cl)Phe-Leu-Arg-DArg-Ile- |
| | Arg-NH₂ |
| 21 | NH₂-Tyr-Sar-(4-Cl)Phe-Leu-Arg-DArg-C₅H₁₀-CONH₂ |
| 22 | NH₂-Tyr-Sar-(4-Cl)Phe-Leu-Arg-DArg-Ile-Arg- |
| | Pro-Lys-NH₂ |
| 23 | NH₂-Tyr-Sar-(4-Cl)Phe-Leu-Arg-DArg-Ile-DArg- |
| | Pro-Dlys-NH₂ |
| 24 | NH₂-Tyr-Sar-(4-C1)Phe-Leu-Arg-DArg-C-H₁₄-CONH₂ |
| 25 | NH₂-Tyr-Sar-(4-Cl)Phe-Leu-Arg-DArg-Ile-DArg- |
| | NH₂ |
| 26 | NH₂-Tyr-Sar-(4-Cl)Phe-Leu-Arg-NMA-Ile-Arg-NH₂ |
| 27 | NH₂-Tyr-Sar-(4-NO₂)Phe-Leu-Arg-NMA-Ile-Arg-NH₂ |
| 28 | NH₂-Tyr-Sar-(4-Cl)Phe-Leu-Arg-DArg-C₇H₁₄-COOH |
| 29 | NH₂-Tyr-Sar-(4-Cl)Phe-Leu-Arg-NMA-C₇H₁₄-CONH₂ |
| 30 | NH₂-Tyr-Sar-(4-NO₂)Phe-Leu-Arg-NMA-C₇H₁₄-CONH₂ |
| 31 | NH₂-Tyr-Sar-(4-NO₂)Phe-Leu-Arg-DArg-C₇H₁₄-CONH₂ |

In particular embodiments, the compound of formula (I) is selected from SEQ ID Nos. 2, 11, 12, 13, 20, 22, 24, 26. 27, 28, 29, 30 and 31.

The peptides of the present invention may be made using methods well known in the art and commercially available amino acid residues. For example, the peptides may be prepared by solid phase synthesis or solution phase synthesis using Fmoc or Boc protected amino acid residues (Jones, Amino Acid and Peptide Synthesis, 1992, Oxford Science Publications and other similar texts).

Also described are peptides in which X₃ is glycine or sarcosine or a hydrophobic amino acid residue having a small side chain, especially where X₃ is glycine, sarcosine, L-alanine, L-valine, L-leucine, L-isoleucine, L-methionine, D-alanine, D-valine, D-leucine, D-isoleucine, D-methionine, more especially where X₃ is glycine, sarcosine, L-alanine, D-alanine, L-valine or D-valine, even more especially where X₃ is glycine or sarcosine, most especially where X₃ is absent or is glycine.

These peptides include:

| SEQ | |
|---|---|
| ID | |
| NO. | Sequence |
| 41 | NH₂-Tyr-Gly-Gly-(4-NO₂)Phe-Leu-Arg-Arg-NH₂ |
| 42 | NH₂-Tyr-Gly-Gly-(4-OCH3)Tyr-Leu-Arg-Arg-NH₂ |
| 43 | NH₂-Tyr-Gly-Gly-(2-NH₂)Phe-Leu-Arg-Arg-NH₂ |
| 44 | NH₂-Tyr-Gly-Gly-Phe-Leu-Arg-His-NH₂ |
| 45 | NH₂-Tyr-Gly-Gly-Phe-Leu-Arg-Asn-NH₂ |
| 46 | NH₂-Tyr-Gly-Gly-Phe-Leu-Arg-Cap-COOH |
| 47 | CH3NH-CH3Tyr-Gly-Gly-Phe-Leu-Arg-Cap-COOH |
| 48 | CH3NH-CH3Tyr-Gly-Gly-Phe-Leu-Lys-Cap-COOH |
| 49 | NH₂-4Py-Gly-Gly-Phe-Leu-Arg-Arg-NH₂ |
| 50 | NH₂-Tyr-Gly-Gly-Phe-Leu-Cit-Arg-NH₂ |
| 51 | NH₂-Tyr-Gly-Gly-Phe-Leu-Asn-Arg-NH₂ |
| 52 | NH₂-Tyr-Gly-D-Ala-Phe-Leu-Arg-Arg-NH₂ |
| 53 | NH₂-Tyr-Gly-Gly-Tal-Leu-Asn-Arg-NH₂ |
| 54 | CH3NH-CH3Tyr-Sar-Gly-Phe-Leu-Arg-Cap-COOH |
| 55 | NH₂-Tyr-Gly-Gly-Phe-Leu-Lys- Cap-COOH |
| 56 | NH₂-Tyr-Gly-Gly-Phe-Leu-Arg-HArg-NH₂ |
| 57 | NH₂-Tyr-Gly-Gly-(4-NO₂)Phe-Leu-Arg-DArg-NH₂ |
| 58 | NH₂-Tyr-Sar-Gly-(4-NO₂)Phe-Leu-Arg-DArg-NH₂ |
| 59 | NH₂-Tyr-Gly-Sar-(4-NO₂)Phe-Leu-Arg-DArg-NH₂ |
| 60 | NH₂-Tyr-Gly-Sar-(4-NO₂)Phe-Leu-DArg-Arg-NH₂ |
| 61 | NH₂-Tyr-Sar-Gly-(4-NO₂)Phe-Leu-DArg-Arg-NH₂ |
| 62 | NH₂-Tyr-Gly-Gly-(4-NO₂)Phe-Leu-DArg-Arg-NH₂ |
| 63 | NH₂-Tyr-Sar-Gly-(4-NO₂)Phe-Leu-DArg-DArg-NH₂ |

### Compositions of the Invention

According to another aspect, the invention resides in a pharmaceutical composition comprising a compound of the invention, or a pharmaceutically acceptable salt, stereoisomer, solvate or prodrug thereof, and a pharmaceutically acceptable carrier, diluent and/or excipient.

Suitably, the pharmaceutically acceptable carrier, diluent and/or excipient may be or include one or more of diluents, solvents, pH buffers, binders, fillers, emulsifiers, disintegrants, polymers, lubricants, oils, fats, waxes, coatings, viscosity-modifying agents, glidants and the like.

The salt forms of the compounds of the invention may be especially useful due to improved solubility.

Diluents may include one or more of microcrystalline cellulose, lactose, mannitol, calcium phosphate, calcium sulfate, kaolin, dry starch, powdered sugar, and the like. Binders may include one or more of povidone, starch, stearic acid, gums, hydroxypropylmethyl cellulose and the like. Disintegrants may include one or more of starch, croscarmellose sodium, crospovidone, sodium starch glycolate and the like. Solvents may include one or more of ethanol, methanol, isopropanol, chloroform, acetone, methylethyl ketone, methylene chloride, water and the like. Lubricants may include one or more of magnesium stearate, zinc stearate, calcium stearate, stearic acid, sodium stearyl fumarate, hydrogenated vegetable oil, glyceryl behenate and the like. A glidant may be one or more of colloidal silicon dioxide, talc or cornstarch and the like. Buffers may include phosphate buffers, borate buffers and carbonate buffers, although without limitation thereto. Fillers may include one or more gels inclusive of gelatin, starch and synthetic polymer gels, although without limitation thereto. Coatings may comprise one or more of film formers, solvents, plasticizers and the like. Suitable film formers may be one or more of hydroxypropyl methyl cellulose, methyl hydroxyethyl cellulose, ethyl cellulose, hydroxypropyl cellulose, povidone, sodium carboxymethyl cellulose, polyethylene glycol, acrylates and the like. Suitable solvents may be one or more of water, ethanol, methanol, isopropanol, chloroform, acetone, methylethyl ketone, methylene chloride and the like. Plasticizers may be one or more of propylene glycol, castor oil, glycerin, polyethylene glycol, polysorbates, and the like.

Reference is made to the Handbook of Excipients 6th Edition, Eds. Rowe, Sheskey & Quinn (Pharmaceutical Press), which provides non-limiting examples of excipients which may be useful according to the invention.

It will be appreciated that the choice of pharmaceutically acceptable carriers, diluents and/or excipients will, at least in part, be dependent upon the mode of administration of the formulation. By way of example only, the composition may be in the form of a tablet, capsule, caplet, powder, an injectable liquid, a suppository, a slow release formulation, an osmotic pump formulation or any other form that is effective and safe for administration.

### Methods of Treatment

Suitably, the pharmaceutical composition is for the treatment of pain.

In another aspect, the invention provides a method of treating or preventing pain in a subject including the step of administering a therapeutically effective amount of a compound of the invention, or a pharmaceutically acceptable salt, stereoisomer, solvate or prodrug thereof, or the pharmaceutical composition comprising a compound of the invention, to the subject to thereby treat or prevent pain.

In a further aspect, the invention provides a use of a compound of the invention, or a pharmaceutically acceptable salt, stereoisomer, solvate or prodrug thereof, in the manufacture of a medicament for the treatment or prevention of pain.

In a yet a further aspect, the invention provides a compound the invention, or a pharmaceutically acceptable salt, stereoisomer, solvate or prodrug thereof, or the pharmaceutical composition comprising a compound of the invention, for use in the treatment or prevention of pain.

As generally used herein, the terms “administering” or “administration”, and the like, describe the introduction of the compound or composition to a subject such as by a particular route or vehicle. Routes of administration may include topical, parenteral and enteral which include oral, buccal, sub-lingual, nasal, anal, gastrointestinal, subcutaneous, intramuscular, intravenous and intradermal routes of administration, although without limitation thereto.

By “treat”, “treatment” or treating” is meant administration of the compound or composition to a subject to at least alleviate, reduce or suppress pain experienced by the subject.

By “prevent”, “preventing” or “preventative” is meant prophylactically administering the formulation to a subject who does not exhibit experience pain, but who is expected or anticipated to likely experience pain in the absence of prevention.

An “effective amount” means an amount necessary at least partly to attain the desired response, or to alleviate, decrease or remove the pain, delay the onset or inhibit progression of the pain, or inhibit the onset of the pain being treated. The amount varies depending upon the health and physical condition of the individual to be treated, the taxonomic group of individual to be treated, the degree of alleviation desired, the formulation of the composition, the assessment of the medical situation, and other relevant factors. It is expected that the amount will fall in a relatively broad range that can be determined through routine trials. An effective amount in relation to a human patient, for example, may lie in the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage. Dosage regimes may be adjusted to provide the optimum therapeutic response. For example, several divided doses may be administered daily, weekly, monthly or other suitable time intervals, or the dose may be proportionally reduced as indicated by the exigencies of the situation. The effective amount and appropriate dosage regimen may be ascertained through routine trial.

As used herein, the terms “subject” or “individual” or “patient” may refer to any subject, particularly a vertebrate subject, and even more particularly a mammalian subject, for whom treatment is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). In a particular embodiment, the subject is a human.

Suitably, the pain being treated is selected from the group consisting of nociceptive pain, inflammatory pain and neuropathic pain, for example, somatic pain, visceral pain, pain syndrome, diabetic neuropathy, trigeminal neuralgia, postherpetic neuralgia, post-stroke pain, complex regional pain syndrome, reflex sympathetic dystrophy, causalgias, cancer pain, surgical pain and psychogenic pain. In some embodiments, the pain is acute pain. In other embodiments, the pain is chronic pain. Any condition for which dynorphin is considered an appropriate treatment or co-treatment may be considered suitable for treatment using a compound of the invention or the composition comprising a compound of the invention.

### EXAMPLES

### Example 1: Synthesis of Peptides

Rink amide AM resin (0.6 mmol/gm original loading) was used as the solid support and Fmoc-protected amino acids were coupled in 4-5 equivalents (relative to original loading) using a coupling reagent cocktail of diisopropylcarbodiimide (DIC) and ethyl (hydroxyamino)cyanoacetate (Oxyma Pure) in 0.5 M concentration in dimethylformamine (DMF). Fmoc deprotection was achieved using 20% piperidine in DMF (60 equivalents) with a 20 minute incubation time. Capping was performed after each amino acid coupling using acetic anhydride (5 M in DMF, 8 eq.) and diisopropyl ethylamine (DIPEA, 2 M in NMP, 8 eq.). After completion of the couplings, the peptide was cleaved from the resin using ≈1.5 mL TFA:TIPS:H₂O cocktail (88:8:4).

SEQ ID NO. 28 was synthesised using the same protocol as above except the first coupling reaction, was performed by tethering to Wang resin (0.38 mmol/gm loading). Wang resin (1 g) was suspended in 9:1 v/v CH₂Cl₂:DMF (≈15 mL). Separately, amino acid and hydroxybenzotriazole (HOBt, 2 eq. of each) were dissolved in a minimum amount of DMF and added to the resin. To this dimethyl aminopyridine (DMAP, 0.1 eq.) and DIC (1 eq.) was added to the resin mixture. The mixture was stirred gently for 3 hours under an inert atmosphere at room temperature. Acetic anhydride (2 eq. and pyridine (2 eq.) was added to the reaction flask and stirred for an additional 30 minutes at room temperature to ‘cap’ any unreacted hydroxyl groups on the resin. The resin was filtered and washed with DMF and dichloromethane (DCM). After synthesis was complete, the peptide was cleaved from the resin using a mixture of DCM:trifluoroacetic acid (TFA):tirisopropylsilane (TIPS) (1:1:0.1; 21 mL), with a cleavage time of 4 hours.

Crude peptides were collected and further purified by preparative HPLC. All peptides were purified using preparative HPLC, Agilent 1200 Chem Station equipped with of binary pump and auto-fraction collector. A Jupiter C₁₈, 10 μm, Proteo 90 Å LC column 250×21.2 mm was used with a flow rate of 10 mL/min. The mobile phase employed was Solvent A: MilliQ water, Solvent B: acetonitrile, both containing 0.1% v/v TFA with a gradient flow 0% to 100% B over 60 min. The peptide purity was determined by using a Shimadzu LC-2040C Nexera-i high-performance liquid chromatography (HPLC) system. The mobile phase employed was Solvent A: MilliQ water, Solvent B: acetonitrile, both containing 0.1% v/v TFA with a gradient flow 0% to 100% B over 35 min. Peptide mass was confirmed by Agilent 1290 ultra-high performance liquid chromatography system coupled with a 6520 accurate mass quadrupole time of flight (Q-TOF) LCMS system.

The peptides synthesized are shown in Table 3:

TABLE 3

| SEQ | | HRMS(M + H)⁺ |
|---|---|---|
| ID | | (M + 2H)⁺/2 |
| NO. | Sequence | (M + 3H)⁺/3 |
| 1 | NH₂-Tyr-Gaba-Phe-Leu-Arg-Arg-NH₂ | 838.87 |
| | | 419.86 |
| 2 | NH₂-Tyr-Sar-Phe-Leu-Arg-Arg-NH₂ | 824.86 |
| | | 412.86 |
| 3 | NH₂-Tyr-Sar-Phe-Leu-Arg-DArg-NH₂ | 412.74207 |
| | | 275.49675 |
| 4 | NH₂-Tyr-Sar-(4-NO₂)Phe-Leu-Arg-DArg-NH₂ | 869.48740 |
| | | 435.24640 |
| 5 | NH₂-Tyr-Sar-(4-NO₂)Phe-Leu-DArg-Arg-NH₂ | 869.47598 |
| | | 412.74836 |
| 6 | NH₂-Tyr-Sar-Phe-Leu-DArg-Arg-NH₂ | 824.49062 |
| | | 412.74836 |
| 7 | NH₂-Tyr-Sar-Phe-Leu-DArg-DArg-NH₂ | 824.49181 |
| | | 412.74861 |
| 9 | NH₂-Phe-Sar-(4-NO₂)Phe-Leu-DArg-DArg-NH₂ | 427.24381 |
| 10 | NH₂-Tyr-Sar-(4-NO₂)Phe-Leu-DLys-DArg-NH₂ | 841.47071 |
| | | 421.23837 |
| 11 | NH₂-Tyr-Sar-(4-Cl)Phe-Leu-Arg-DArg-NH₂ | 429.72790 |
| 12 | NH₂-Tyr-Sar-(4-F)Phe-Leu-Arg-DArg-NH₂ | 842.47212 |
| | | 421.73863 |
| 13 | NH₂-Tyr-Sar-(4-Cl)Phe-Leu-NMA-DArg-NH₂ | 436.76510 |
| 14 | NH₂-Tyr-Sar-(4-F)Phe-Leu-NMA-DArg-NH₂ | 428.74938 |
| 15 | NH₂-Tyr-Sar-(4-NO₂)Phe-Leu-NMA-DArg-NH₂ | 883.48741 |
| | | 442.24612 |
| 16 | NH₂-Tyr-Sar-(4-Cl)Phe-Leu-Lys-DArg-NH₂ | 830.44251 |
| | | 415.72677 |
| 17 | NH₂-Tyr-Sar-(4-Cl)Phe-Leu-Arg-NMA-NH₂ | 872.52423 |
| | | 436.76557 |
| 18 | NH₂-Tyr-Sar-(4-NO₂)Phe-Leu-Arg-NMA-NH₂ | 883.46221 |
| | | 442.22059 |
| 19 | NH₂-Tyr-Sar-(4-NO₂)Phe-Leu-NMA-NMA-NH₂ | 897.47737 |
| | | 449.22867 |
| 24 | NH₂-Tyr-Sar-(4-Cl)Phe-Leu-Arg-DArg- | 500.28418 |
| | C₇H₁₄-CONH₂ | 333.85961 |
| 26 | NH₂-Tyr-Sar-(4-Cl)Phe-Leu-Arg-NMA- | 571.30413 |
| | Ile-Arg-NH₂ | 381.19815 |
| 27 | NH₂-Tyr-Sar-(4-NO₂)Phe-Leu-Arg-NMA- | 576.81570 |
| | Ile-Arg-NH₂ | 384.87264 |
| 28 | NH₂-Tyr-Sar-(4-Cl)Phe-Leu-Arg-DArg- | 501.95772 |
| | C₇H₁₄-COOH | 334.29146 |
| 29 | NH₂-Tyr-Sar-(4-Cl)Phe-Leu-Arg-NMA- | 507.30050 |
| | C₇H₁₄-CONH₂ | 371.28080 |
| 30 | NH₂-Tyr-Sar-(4-NO₂)Phe-Leu-Arg-NMA- | 512.80625 |
| | C₇H₁₄-CONH₂ | 342.20677 |
| 31 | NH₂-Tyr-Sar-(4-NO₂)Phe-Leu-Arg-DArg- | 505.79909 |
| | C₇H₁₄-CONH₂ | 337.53506 |

### Example 2: cAMP Assay

Cyclic AMP (cAMP) production occurs as a biological consequence of pain and inflammation, opioid agonists inhibit this production through a receptor specific mechanism and as such is viewed as the industry ‘gold-standard’ for assessment of opioid potency. It is a surrogate measure for the analgesic properties of opioids in cell-based assays. Highly potent compounds, like morphine or fentanyl, for example, have strong inhibitory effects on cAMP production at low nanomolar (nM) concentrations. Thus, cAMP assays serve as a measure of the analgesic potential of test compounds, as compared to control/reference compounds of high potency and selectivity.

The cAMP assay was performed according to the manufacturer's instruction (ALPHAScreen cAMP kit, Perkin Elmer). HEK 293 cells that stably overexpress Kappa opioid receptor (KOR), Delta opioid receptor (DOR) and Mu opioid receptor (MOR) were grown to approximately 90% confluence in a 75 mm² flask using standard culture techniques (DMEM, 10% foetal bovine serum). Cells were harvested using EDTA solution and diluted to 4 million cells per mL in HBSS buffer containing 0.1% bovine serum albumin (BSA), 5 mM HEPES buffer and 0.5 mM 3-isobutyl-1-methylxanthine (iBMX), as per manufacturer's instruction. In a white 96 well ½ volume plate, cells (20 μL, 80K cells containing acceptor beads 0.25%) were added to 20 μL forskolin (50 μM, in above HBSS buffer) containing the test compound at the given concentration (final 10 μM-1 pM) and incubated at 37° C. for 1 hour. CR845 (SEQ ID NO. 35) was used as a positive control with high potency, selectivity and stability as reported in Menzaghi et al., 2015, The Journal of Pain, 16(4), S81.

Cell reactions were stopped using lysis buffer, as per manufacturer's instructions (0.1% BSA, 5 mM HEPES, 0.3% Tween-20™ in water, pH 7.4 containing 0.25% donor beads and 0.5% cAMP tracer, 60 μL). Plates were left at room temperature in a dark humidity box on a rotary shaker table overnight and on a fluorimeter (Perkin Elmer EnSight) using ALPHAScreen relevant filters.

The results are shown in Table 4:

TABLE 4

| SEQ ID | KOR | | DOR | | MOR | |
|---|---|---|---|---|---|---|
| No. | 10 μM | 1 μM | 10 μM | 1 μM | 10 μM | 1 μM |
| 1 | 22.91 | 4.23 | 19.55 | 2.57 | 7.28 | −6.78 |
| 2 | 32.41 | 21.65 | 21.96 | −0.47 | 12.41 | −2.5 |
| 3 | 43.58 | 27.24 | 2.24 | −2.27 | 2.17 | −0.68 |
| 4 | 40.29 | 34.93 | 2.04 | −0.75 | 2.52 | 0.17 |
| 5 | 43.14 | 33.44 | 2.02 | 0.05 | 4.32 | −0.05 |
| 6 | 35.13 | 14.22 | 2.25 | −0.03 | 7.84 | 1.04 |
| 7 | 36.03 | 18.27 | 2.53 | −0.68 | 4.19 | 0.13 |
| 9 | 23.06 | 2.46 | 1.83 | −0.5 | −0.64 | −0.97 |
| 10 | 25.11 | 8.47 | −0.95 | −3 | 0.02 | −1.27 |
| 11 | 33.99 | 27.7 | 4.27 | −1.18 | 1.37 | −0.86 |
| 35 (CR845) | 34.61 | 25.93 | 3.27 | −5.72 | 8.07 | 0.37 |

### Example 3: Cyclic AMP Assays on Potent Peptide Compounds

The assay of Example 2 was repeated at 8 concentrations with peptide derivatives of higher potency as well as further positive and negative controls. The peptide derivatives tested were SEQ ID Nos. 2 to 4, 11 to 20, 24, 25 and 28 to 31 and Dyn 1-17. CR845, referred to as SEQ ID NO. 35 (DPhe-DPhe-DLeu-DLys-Cap-CO₂H), was used for a positive control selective for KOR. SP12 (SEQ ID NO. 36: 2-NH₂-Phe-Phe-Leu-Gly-Arg-Arg) was selected as a negative control, having no measurable potency at 1 μM in any opioid-receptor cell type. Small molecule agonist compounds U50488H (KOR selective, Von Voigtlander and Lewis, Progress in Neuro-Psychopharmacology and Biological Psychiatry, 1982, 6(4), 467-470), SNC80 (DOR selective, Metcalf et al., ACS Chemical Neuroscience, 2012, 3(7), 505-509) and fentanyl (MOR selective, Suzuki and El-Haddad, Drug and Alcohol Dependence 2017, 171, 107-116) and endogenous dynorphin 1-17 (Dyn1-17, SEQ ID NO. 37: Tyr-Gly-Gly-Phe-Leu-Arg-Arg-Ile-Arg-Pro-Lys-Leu-Lys-Trp-Asp-Asn-Gln) were used as positive controls for KOR, DOR and MOR cells. The compounds were assessed over 8 concentrations ranging from 10 μM to 1 pm (10-fold serial dilution) in 96 well plate format. All three cell types were assessed simultaneously and each plate included Dyn1-17 (SEQ ID NO. 37) and the respective small molecule agonist. The concentration response curves were constructed in two ways, with low efficacy derivatives being examined on 2 independent occasions and all others being assessed on at least 3 independent occasions. The results being an average of two or more independent experiments.

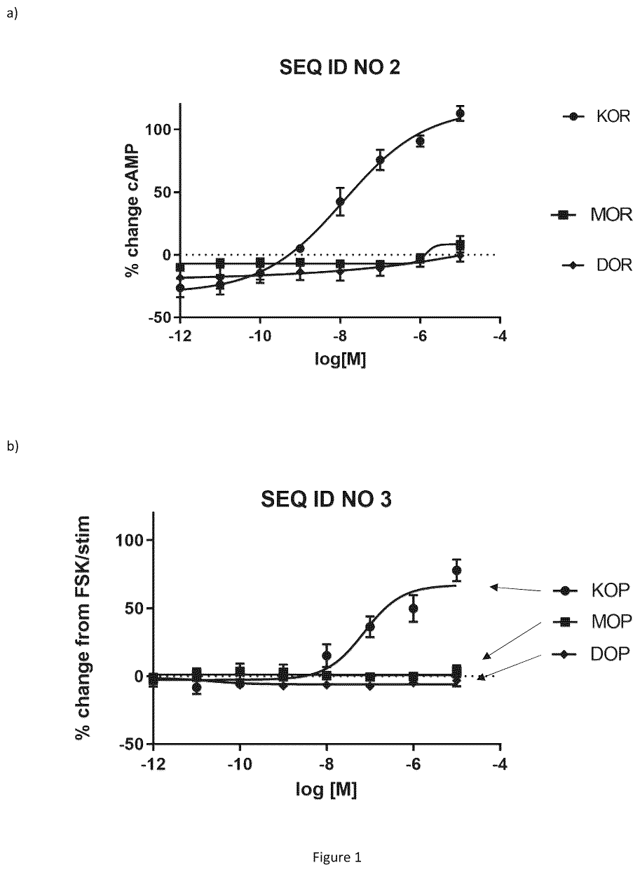

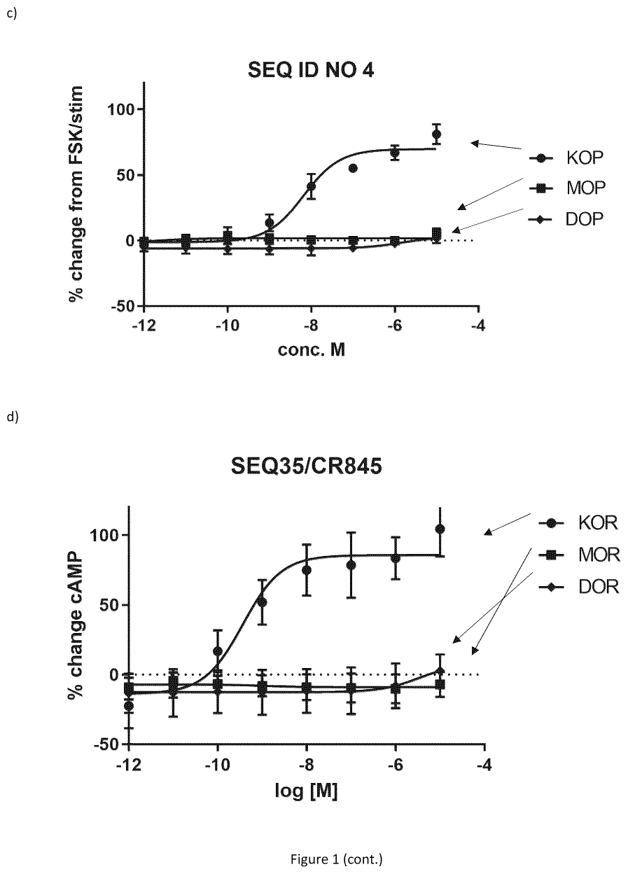

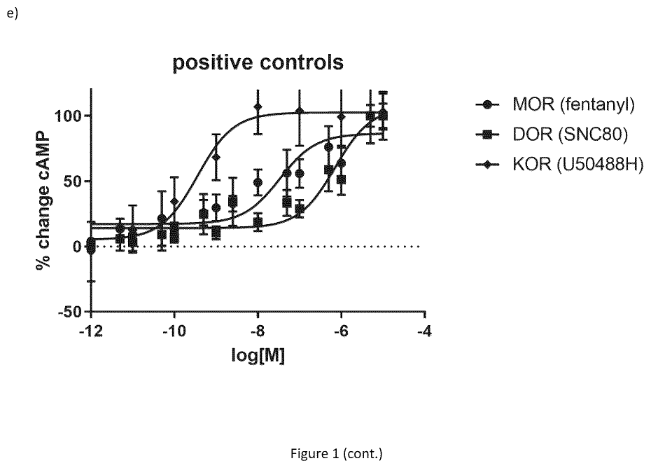

The results for SEQ ID Nos 2, 3 and 4 together with SEQ ID NO. 35 (CR845) and positive controls are shown in FIG. 1. EC₅₀ values are shown in Table 5.

TABLE 5

| | Activity (EC50, nM) | | |
|---|---|---|---|
| SEQ ID NO. | KOR | DOR | MOR |
| 2 | 8.6 | >10000 | 6700 |
| 3 | 74.7 | >10000 | >10000 |
| 4 | 8.4 | >10000 | >10000 |
| 11 | 6.1 | >10000 | >10000 |
| 12 | 6.7 | >10000 | 3448 |
| 13 | 4.54 | >10000 | >10000 |
| 14 | 11.63 | 1662 | 3997 |
| 15 | 13.5 | 1320 | >10000 |
| 16 | 26 | n.t. | >10000 |
| 17 | 3.9 | — | >10000 |
| 18 | 1.77 | >10000 | >10000 |
| 19 | 31.05 | >10000 | >10000 |
| 20 | 2.05 | 3584 | >10000 |
| 24 | 3.87 | >10000 | >10000 |
| 25 | 2.13 | >10000 | 1611 |
| 28 | 2.33 | >10000 | >10000 |
| 29 | 3.79 | >10000 | >10000 |
| 30 | 4.45 | >10000 | >10000 |
| 31 | 2.49 | >10000 | >10000 |
| 35 (CR845) | 0.37 | >10000 | >10000 |
| 38 (Dyn1-7) | 1600 | 870 | 3738 |
| 37 (Dyn1-17) | 3.15 | >10000 | 4400 |
| U50488H | 7.8 | N/A | N/A |
| Morphine | N/A | N/A | 27.3 |
| Fentanyl | N/A | N/A | 33.7 |
| SNC80 | N/A | 11.7 | N/A |

### Example 4: cAMP Activity in the Presence of Naloxone

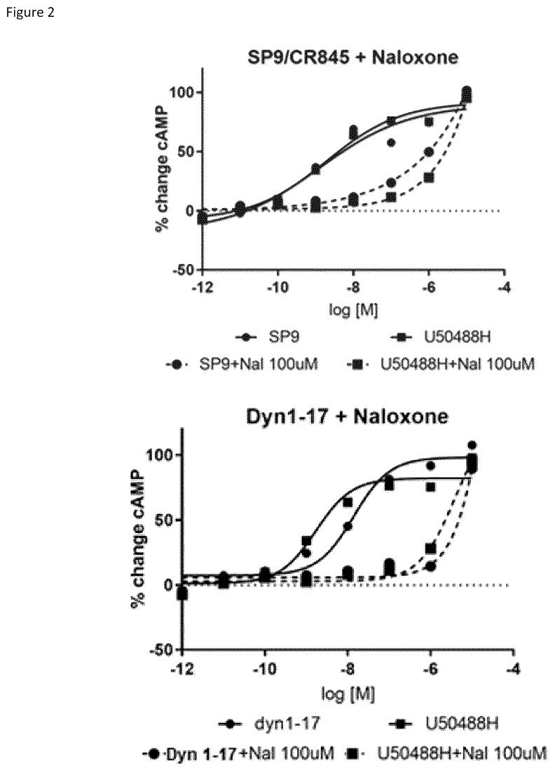

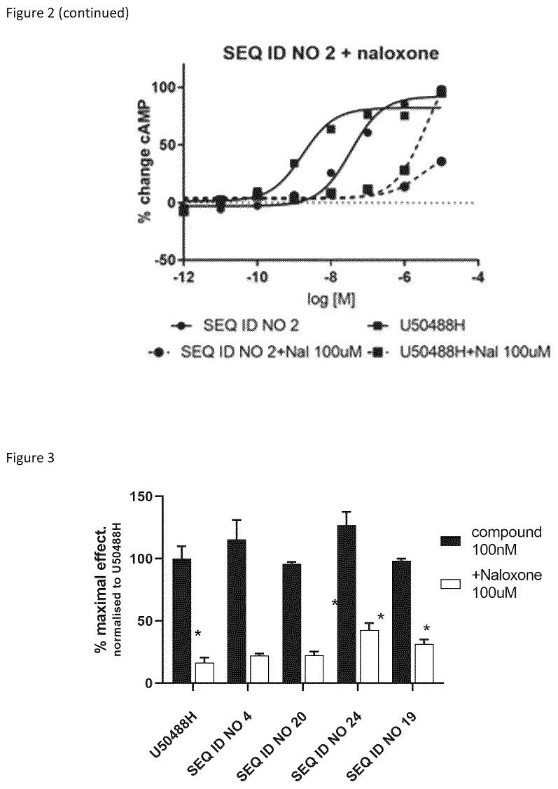

SEQ ID Nos. 2, as well as Dyn1-17 (SEQ ID NO. 37) and the respective small molecule agonists U50488H and CR845 (SEQ ID NO. 35), were tested against non-selective opioid antagonist naloxone (100 μM) to establish whether the cAMP modulatory effect was specifically induced by the respective opioid receptors. The results are shown in FIG. 2.

This experiment was repeated with SEQ ID Nos. 4, 7, 11 and 16. The results are shown in FIG. 3.

Naloxone was found to abolish all activity of all compounds assessed in all cell lines, definitively confirming the specificity of the peptides at each opioid receptor in KOR, MOR and DOR cell lines.

### Example 5: Stability

The stability of the peptides was analysed in typsin, plasma and water. Stability of linear peptides is generally low due to rapid degradation by endogenous peptidases and proteases. While stability in bodily fluids is important, absolute stability may not be prudent or desirable due to potential build-up in vivo leading to adverse side effects, and/or hazards to the environment and recirculation of exceptionally stable compounds in waterways. Thus, compounds that are extremely stable in either plasma or typsin are not generally considered “drug-like”.

Stability in bovine trypsin was analysed for SEQ ID Nos. 2 to 7 and 11 to 17. Peptides were added to trypsin solution (bovine pancreatic trypsin 2.5 μg/mL in NH₄HCO₃ buffer, pH approximately 8-8.5, 37° C.) in a water bath with final concentrations of 100 μM peptide. A 50 μL sample was taken immediately and precipitated in 150 μL of cold acetonitrile (9:1 acetonitrile:water) This sample became the baseline, or t=0 min sample. The trypsin peptide mixture was immediately returned to the water bath and subsequent 50 μL samples were taken at 5, 10, 15, 30, 60 and 120 min. At each time point, the 50 μL sample was collected and immediately added to cold acetonitrile (ACN). Each sample was vortexed for 30 sec and then centrifuged at room temperature (RT) (13K rpm, 5 min). 150 μL of the supernatant was taken and directly place in a glass HPLC vial for LCMS analysis.

Stability in plasma was assessed in rat plasma. Plasma was collected in-house from adult mixed-gender Wistar rats, prepared using 2% EDTA as per standard practice. Peptides were added to the rat plasma samples at 37° C. in a water bath with final concentrations of 100 μM (1:9 peptide in water:plasma). The assay was then carried out in the same manner as the assay in bovine trypsin.

Stability in water was carried out by incubating each candidate in ultra-pure, Milli-Q water (Millipore Corporation). Again, volumes, times and preparation protocols were exactly as described above in plasma stability method above.

The results are shown in Table 6:

TABLE 6

| | Half Life (minutes | | |
|---|---|---|---|
| SEQ ID NO | Trypsin | Plasma | Water |
| 2 | 1.8 | 96.7 | >1000 |
| 3 | 13.6 | 72 | n.t |
| 4 | 15.4 | 71.01 | n.t. |
| 5 | 3250.0 | 28.69 | n.t. |
| 6 | 1265.0 | n.t. | n.t. |
| 7 | 587.0 | n.t. | n.t. |
| 11 | 15.39 | n.t. | n.t. |
| 12 | 13.7 | 31.9 | n.t. |
| 13 | 276 | 239.4 | n.t. |
| 14 | 1627 | 323.9 | n.t. |
| 15 | 12.1 | 168.2 | n.t. |
| 16 | 69.9 | 69.9 | n.t. |
| 17 | >1000 | 174.1 | n.t. |
| 38 (Dyn1-7) | <1.0 | 8.1 | >1000 |
| 35 (CR845) | 1055.0 | 648.6 | >1000 |

### Example 6: Binding Efficiency Assay for KOR Binding

Binding efficiency was determined using a commercial Homogenous Time-Resolved Fluorescence (HTRF) assay specific for KOR-binding studies (Tag-Lite® Opioid KOP Receptor Ligand Binding Assay, CisBio). Experimental protocols were as according to the manufacturer's instruction. Plates were read on a Tecan Spark Fluorimeter. Data was analysed using a combination of Microsoft Excel and Graphpad Prism software packages. The K_{d}, IC₅₀ and Kᵢ were determined in Prism.

### Example 7: Desensitisation Assay

Methods are similar to previously described by Koch et al (J. Biol. Chem., 2001, 276(3): 31408-14). Two 25 cm² flasks of HEK293-KOPr cells per compound to be tested were grown to confluence (DMEM/10% FBS). Media was replaced with fresh warmed sterile media (DMEM/10% FBS) in both flasks, one containing the test peptide or reference/control compound (1 μM) the other acting as control (untreated). Flasks were returned to the incubator for 6 hours, following which cells were harvested and tested for cAMP modulation using the same kit and protocol as in Example 3. Cells were treated with the same peptide/compound to which they were pre-treated in a concentration curve manner, as described above. Data was compared for EC₅₀ with and without pre-treatment.

SEQ ID NOs 4, 7, 11 and 16 along with U50488H, Dyn1-7 (SEQ ID NO. 38: Tyr-Gly-Gly-Phe-Leu-Arg-Arg) and Dyn1-17 were (SEQ ID NO. 37) screened for binding affinity to KOR (Ki relative to K_{d} of naltrindole) using a commercial Homogenous Time Resolved Fluorescence (HTRF) assays. Morphine was used as a negative control for KOR binding. The K_{d} of the reference ligand (Naltrindole; supplied in HTRF kit) was determined to be 0.8 nM (n=3). Test compounds were assayed for competitive binding against naltrindole (8 nM) in order to determine their Ki values. Calculated Ki values relative to naltrindole are shown in Table 7. All test peptides assayed had Ki values in the low-to-mid nanomolar range comparable to Dyn1-17 (SEQ ID NO. 37), Dyn1-7 (SEQ ID NO. 38) control peptides and U50488H with a similar rank order as the EC₅₀. As expected, morphine displayed poor affinity for KOR.

TABLE 7

| Compound Tested | Log IC₅₀ | IC₅₀ (nM) | Ki (nM) |
|---|---|---|---|
| Morphine | −4.91 | 12240.0 | 1130.4 |
| U50488H | −6.07 | 857.9 | 79.2 |
| SEQ ID NO. 37 | −6.78 | 165.7 | 15.3 |
| Dyn1-17 | | | |
| SEQ ID NO. 38 | −6.60 | 253.5 | 23.4 |
| Dyn1-7 | | | |
| SEQ ID NO. 4 | −7.52 | 30.2 | 2.8 |
| SEQ ID NO. 20 | −6.54 | 288.2 | 26.6 |
| SEQ ID NO. 24 | −7.14 | 72.6 | 6.7 |
| SEQ ID NO. 19 | −5.71 | 1953.0 | 180.4 |

### Example 8: Involvement of KOR Agonist Peptides in Receptor Desensitisation

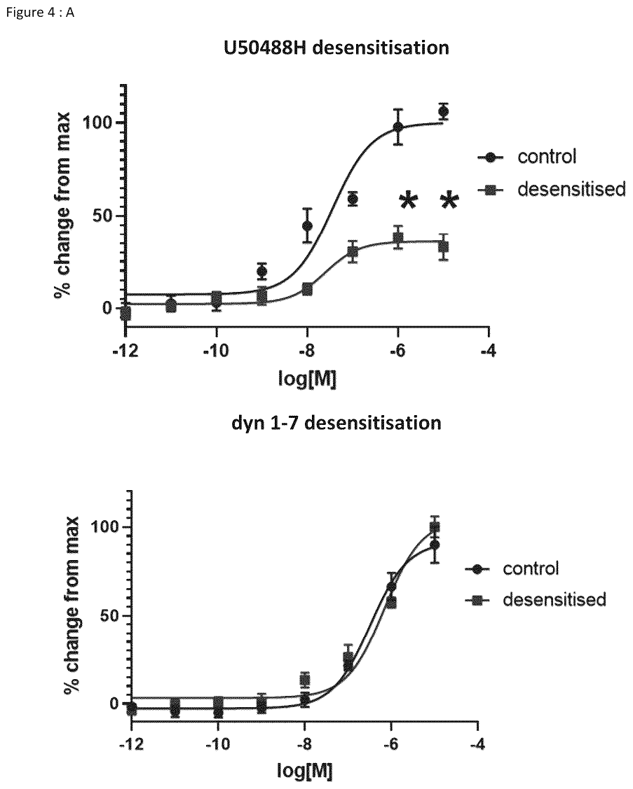

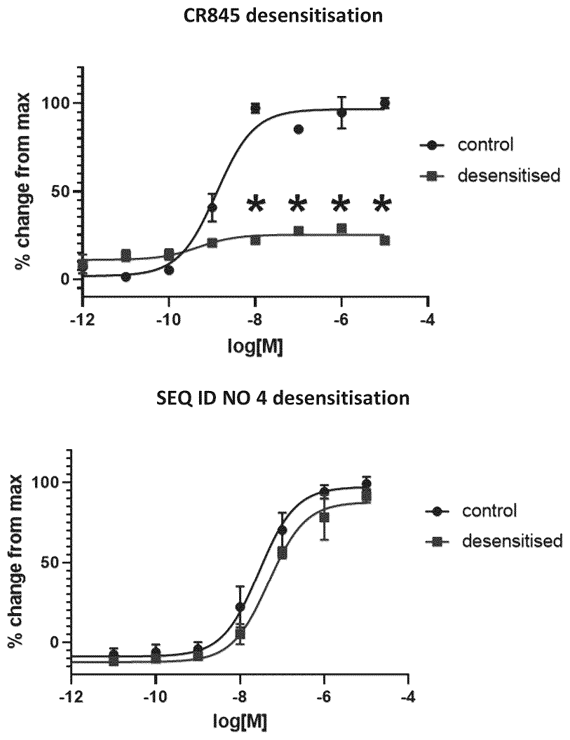

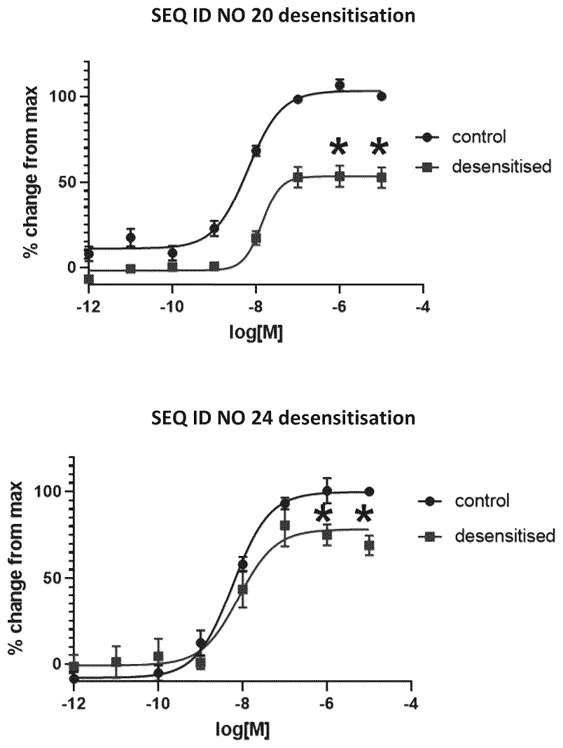

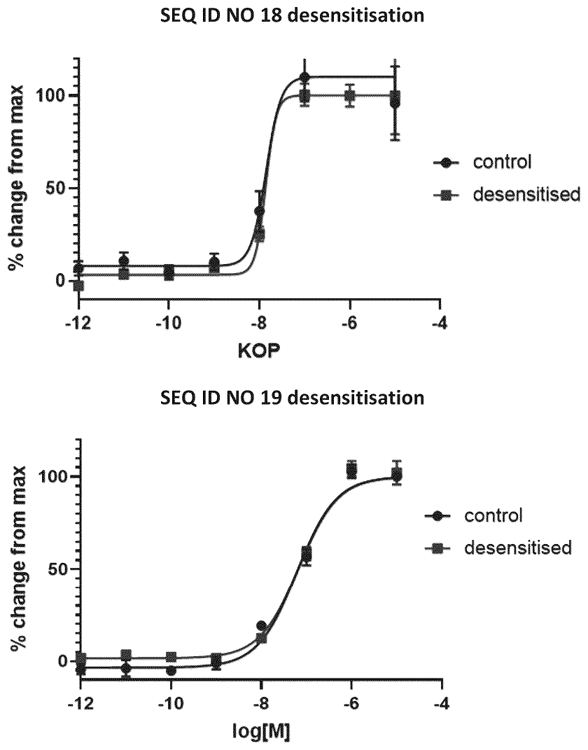

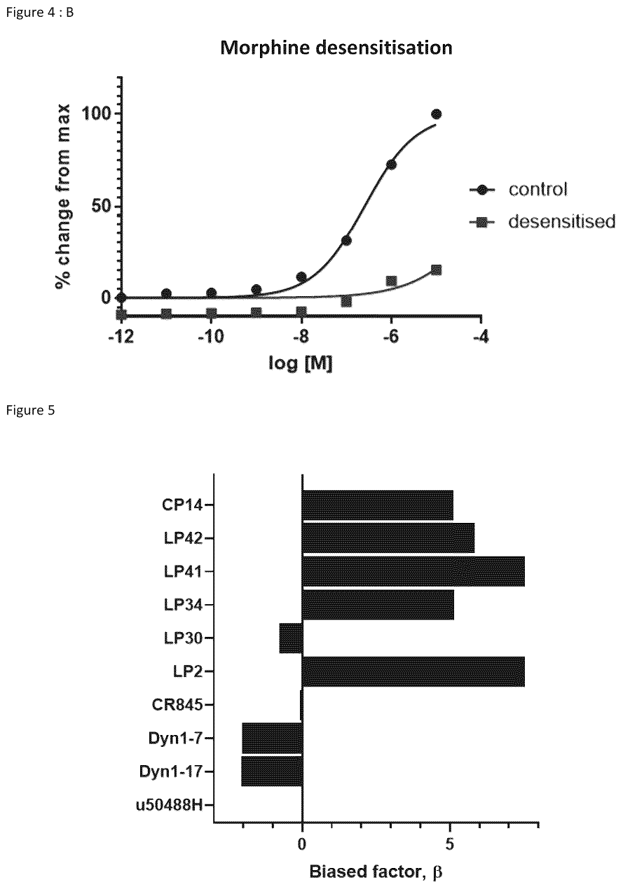

Peptides were screened for KOPr desensitisation in HEK293-KOPr cells, similar to previously described in Koch, T.; Schulz, S.; Pfeiffer, M.; Klutzny, M.; Schroder, H.; Kahl, E.; Hollt, V., J Biol Chem 2001, 276 (33), 31408-14. Pretreatment of cells with reference compounds (1 μM) U50488H or CR845 compound for 6 hours resulted in a significant reduction maximal effect (cAMP % change; unpaired two tailed ttest p<0.0001) indicative of receptor desensitisation (FIG. 4). Dyn1-7 did not show any desensitisation with superimposable concentration response curve with or without peptide pretreatment. Of the test peptides, SEQ ID No. 20 showed a desensitisation characterised by a significant 50% (approx.) reduction in maximal response (unpaired two tailed ttest p<0.0001) similar to U50488H. SEQ ID 24 also showed modest desensitisation which also reached significance (p<0.005), however the extent the maximal response was reduced was less than that observed for CR845, U50488H and SEQ ID NO. 20. SEQ ID Nos 4, 18 and 19 showed no sign of desensitisation, displaying identical sensitivity to the peptides even after 6 hours of treatment. Given their metabolic stability is high, it is unlikely the lack of desensitisation is due to a rapid loss of activity caused by metabolic degradation in the assay buffer, and it can be predicted that, similar to Dyn1-7, SEQ ID Nos 4, 18 and 19 do not cause KOR desensitisation in these cells with respect to cAMP signalling modulation. As a control for the assay, morphine was tested for desensitisation in HEK293-MOPr cells in the same manner. Similar to U50488H in KOR Morphine pre-treatment caused a predictable strong desensitisation of MOR (FIG. 4).

### Example 9: pERK Activation for Bias Signaling Determination

Protocols are as recommended by the pERK kit manufacturer's instruction (PerkinElmer AlphaLISA SureFire Ultra pERK_{1/2} assay kit) with minor modifications. Briefly, HEK293-KOPr cells were grown to confluence and prepared at 200K cells/mL using 0.25% EDTA. Cells were seeded in DMEM/10% FBS, 40 k/well, in sterile black, clear bottom, 96 well plates (one plate per compound). The outer most wells were omitted to avoid edge effects. Cells were returned to the incubator with the lid on the plate and left for 48 hours. Old media was replaced with 40 μL clear (indicator dye and FBS free) prewarmed HBSS and incubated for 20 min at 37° C. Serially diluted test peptide (6×10 fold dilutions; final concentration in well 1-10 μM in HBSS) was added (10 μL) to each respective well at various time points (30, 20, 10, 5 and 0 minutes). Assay was stopped using the provided lysis buffer, following which plates were placed on a shaker for 10 min at room temperature, the plate was centrifuged (3700 rpm, 10 min) and the supernatant was collected. 5 μL of lysate from each assay well was added to a well of a white 384 well plate, to which 2.5 uL of commercial ‘acceptor bead’ reaction media was added and the plate left in dark at room temp for 1 hour. 2.5 μL of commercial ‘donor bead’ reaction media was added to each well, and the plate left to incubate in a humidity box at room temperature. Plates were read for ALPHA signal using a PerkinElmer Ensight Fluorimetric Plate Reader running Kalaido software. Using this method, pERK_{1/2} production was recorded in a time and concentration dependent manner. Total ERK was not measured. A bias factor was calculated from the cAMP and pERK data, as previously reported by Rajagopal et al., (Mol. Pharmacol., 2011, 80(3): 367-77), using the following formulae;

$${ε}_{ref . lig} = \frac{{E}_{max}}{{EC}_{50}}$$

$${σ}_{response} = log ( \frac{{ε}_{lig}}{{ε}_{ref}} )$$

$${β}_{lig} = \frac{( {σ}_{response 1} {\_σ}_{response 2} )}{√ 2}$$

where ε is the efficiency coefficient for reference (ref) OR test ligand (lig), σ is the relative response/pathway-specific signal factor and β is the pathway signal bias factor. Response 1 was cAMP, response 2 was pERK. Eₘₐₓ (maximal effect recorded per compound) and EC₅₀ were derived from the concentration response curves of cAMP and pERK activity respectively.

Desensitisation has been linked to receptor/G-protein uncoupling and internalisation events, thus it is possible these peptides behave differently as agonists than other molecules such as U50488H. The activation of MAP kinases (such as phosphorylated extracellular regulated kinase 1 and 2 (pERK_{1/2})) by G-protein coupling are second messengers often linked to β-Arrestin recruitment, receptor internalisation, recycling and desensitisation. Furthermore, many studies have speculated an involvement for MAPKs like pERK_{1/2} in the development of tolerance. Thus, there is an intriguing possibility that the lack of acute desensitisation observed within our peptide library may involve a bias-signalling component at KOPr, whereby pERK_{1/2} activation is reduced, if not avoided.

SEQ ID Nos 4, 18, 19, 20 and 24 and control compounds U50488H, CR845 (SEQ ID NO. 35), Dyn 1-7 (SEQ ID NO. 38) and Dyn 1-17 (SEQ ID NO. 37) were tested in a quantitative pERK1/2 assay in a time and concentration dependent manner. U50488H, CR845 (SEQ ID NO. 35), Dyn1-7 (SEQ ID NO. 38) and SEQ ID NO. 20 all caused time and concentration dependent pERK expression in HEK293-KOPr cells. Expression of pERK was transient and short lived, indicative of early-phase pERK release. SEQ ID NO 24 showed some influence over pERK induction, however only at higher concentrations, not having a classical sigmoidal dose-response curve. SEQ ID Nos 4, 18 and 19, all failed to show any pERK activity. An EC₅₀ and maximal response (Eₘₐₓ) for pERK was derived, and a bias agonist factor was calculated, using U50488H as a reference compound. FIG. 5 shows the relative bias factor (β) for each peptide/compound. Negative β values indicate bias for pERK activation, positive β values indicate bias for cAMP modulation, relative to U50488H respectively. As predicted from the lack of pERK activity, SEQ ID Nos 4, 18 and 19, all showed bias towards cAMP relative to U50488H. Interestingly, SEQ ID NO 24, which showed some pERK activity at higher concentrations also had a bias towards cAMP. Dyn1-7 showed clear bias towards pERK, whereas SEQ ID NO 20 had a marginal bias for pERK. CR845 had no bias either way, showing identical signal preference to U50488H. From these data it is perhaps apparent that the compounds with the greatest potential for desensitisation (FIG. 4) also showed highest propensity for pERK activation and thus a negative bias factor, indicating there may be a relationship between desensitisation and pERK in KOR signalling.

### Example 10: Animal Study of Inflammatory Nociception

Ethical approval for all in vivo experimental protocols was obtained from the University of Queensland Health Sciences Ethics Committee. All procedures adhere to The Australian Code of Practice for Use Of Animals for Scientific Purposes (2013) and reported in accordance with the ARRIVE guidelines (Kilkenny et al., Br. J. Pharmacol., 2010, 160(7), 1577-1579). Wistar rats were obtained from the Australian Animal Resource Centre (Canning Vale, WA) and transported by Australian standard air and road methods. Animals were housed 3 per box at appropriate temperature/pressure environments in a 12 h light/dark cycle according to the standard of holding facility, with food and water provided ad libitum. At least 48 h habituation in the housing facility was provided prior to any experimental intervention. After experimentation, rats are euthanized by appropriate and approved means (CO₂ inhalation).

### Freund's Complete Adjuvant Model of Inflammatory Nociception in Rats

Experiments are similar to those previously performed (Morgan et al., Peptides, 2017, 89: 9-16). On day 1, rats were weighed and health checked, and baseline paw withdrawal (Randall-Selitto apparatus) and paw volume (plethysmometer) measurements made of both the left and the right hind paws. Rats were then lightly anaesthetised using isoflurane inhalation, and 150 μL of Freund's Complete Adjuvant (FCA) injected subcutaneously to the left paw pad. The right paw received no treatment. Rats were returned to their home cages and allowed to recover. On day 5, both hind paws of each rat were measured for paw withdrawal and volume (t=0). Rats were again lightly anaesthetised, and test compound (0.01-10 mg/kg, 50 μL in sterile isotonic injectable saline) injected subcutaneously into the left affected paw pad. Rats were allowed to recover. Paw pressure measurements (force in grams) to illicit a withdrawal reaction were made by Randall-Selitto methods in both hind paws at t=15, 30, 60 and 120 minutes post compound administration. U50488H (0.17 mg/kg) was used as a KOR reference compound, and morphine (0.3 mg/kg) and fentanyl (0.001-0.003 mg/kg) were used as MOR-active clinically relevant reference compounds. Saline vehicle acted as a negative control. Paw volume measurements were made at t=0 and t=120 min using a plethysmometer (Ugo-Basile Italy). Data was analysed using a combination of Microsoft Excel and Graphpad Prism software packages.

An efficacy index (Iₑ) was calculated to represent efficacy relative to administered dose (in molar), as normalise to a comparator compound. The following formula was used;

$$Efficacy Index ( {I}_{e} ) = - ( log ( D * {A}_{t} ) ) - {A}_{o}$$

where D is dose administered (in Molar), Aₜ is the area under curve of the test peptide and Aₒ is the area under the curve for the control/reference compound, morphine.

Naloxone methiodide was used in the FCA model to antagonise opioid receptor function. Naloxone was administered intraplantar (i.pl, 50 μL 1 mg/kg in saline, to the inflamed paw) following the baseline measurements, under isoflurane anaesthesia 15 minutes prior to i.pl peptide administration. The experimental procedure for the FCA model did not vary from that described above thereafter.

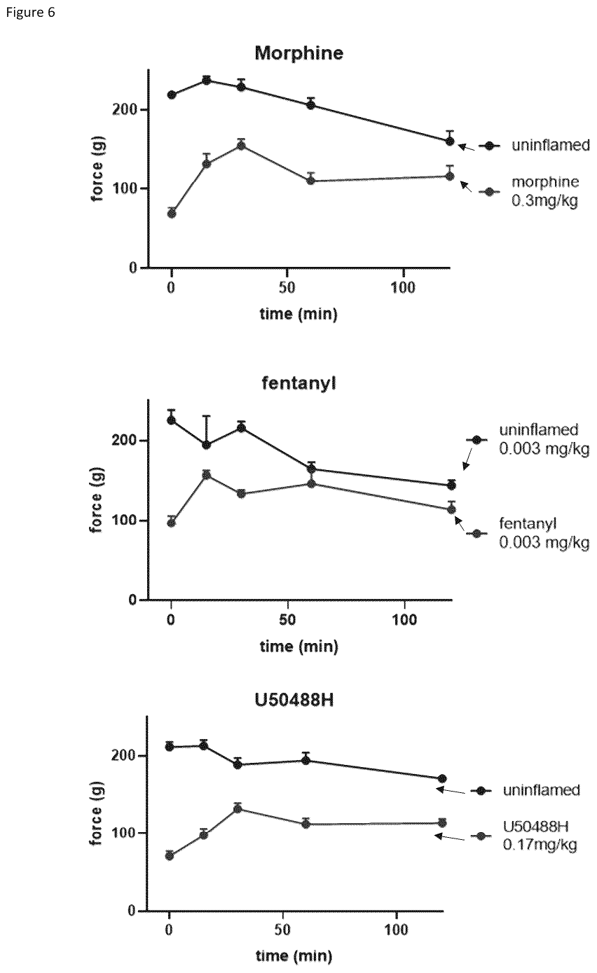

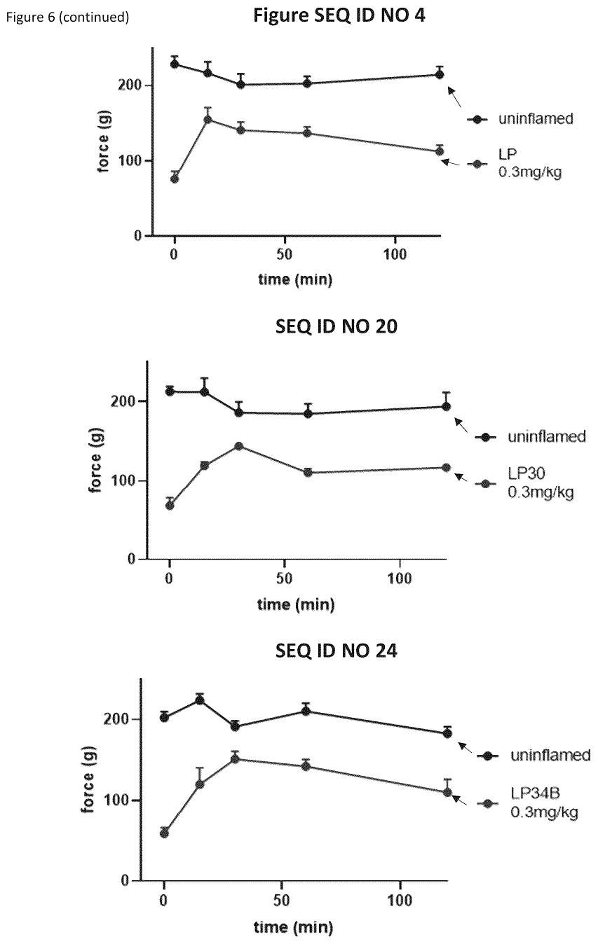

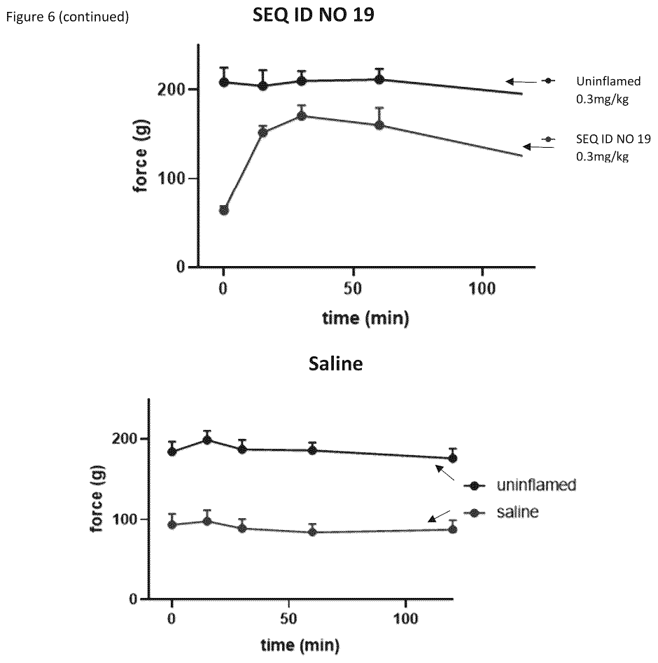

SEQ ID Nos 4, 18, 19, 20 and 24 all proved to have relevant drug-like attributes with respect to low nanomolar potency, high selectivity for KOR over other ORs, favourable metabolic stability in trypsin and plasma and low nanomolar KOR binding coefficients. With the exception of SEQ ID NO 20, all showed a relative bias towards cAMP modulation in HEK293-KOPr cells and very little potential for desensitisation in vitro. These peptides were considered the most promising drug-like candidates, and consequently selected for further testing in vivo in the Freund's Complete Adjuvant (FCA) model of inflammatory pain. FCA administration caused a significant oedema whereby the affected paw swells to around twice its original size (data not shown) which was associated with a predicted decrease in paw withdrawal threshold, indicative of hyperalgaesia. Administration of KOR agonist U50488H (0.17 mg/kg i.pl) directly to the affected paw caused an increase in paw withdrawal threshold (in grams) closer to baseline levels, characteristic of an opioid-like antinociceptive effect (FIG. 6). This effect was short-lived, lasing for no longer than 60 min, returning to the original withdrawal threshold prior to compound administration by 120 min.

Interestingly, all three reference compounds morphine, fentanyl and U50488H showed effects in the contralateral (uninflamed and untreated) control paw by 120 minutes following its administration into the ipsilateral paw (indicated by the black arrow in FIG. 9), whereby the paw withdrawal threshold was significantly reduced compared to baseline (time point t=0, p>0.05 paired T-test). This apparent acute allodynia-inducing effects was not observed for saline control nor any of the test peptides.

Area under the curve (AUC) of each compounds' effect in vivo was calculated as a surrogate measure for overall efficacy. Individual peptides were compared to control compounds (U50488H, morphine (0.3 mg/kg), and fentanyl (3.3 μg/kg)). SEQ ID Nos 4 and 19 (each 0.3 mg/kg, 2.0 mM and 2.07 mM, respectively) showed significantly better efficacy than U50488H. (0.17 mg/kg, 2.7 mM when AUC were compared using Mann-Whitney t-tests (p<0.05) at the given doses.

An Efficacy Index (Iₑ) was calculated for each peptide/compound giving a relative score of effectiveness per unit of molar dose. Fentanyl showed a very high Iₑ simply due to the exceptionally low dose administered (60 μM). SEQ ID Nos 19 and 24 showed statistically significantly greater Iₑ over morphine (Kruskal-Wallis ANOVA; morphine is reference therefore Iₑ is 0). All other compounds were not statistically different to morphine Iₑ. SEQ ID Nos 19 and 24 which showed significantly improved Iₑ over morphine were tested for opioid-receptor specificity in the FCA model.

Naloxone pre-treatment effectively blocked the activity of each peptide, clearly indicating the anti-nociceptive effects were opioid receptor-mediated.

### Example 11: Evaluation of Pharmacokinetic Parameters

Basic pharmacokinetic (PK) parameters and the potential for systemic delivery was determined for the most promising compounds demonstrating efficacy interrogated in the FCA model. Methods are as previously reported (Hill et al., ACS Med. Chem. Lett. 2014, 5(10), 1148-51; Lohman et al., Chem. Commun. (Camb), 2019, 55(89), 13362-13365; Neilsen et al., Org. Lett., 2012, 14(22), 5720-3; Neilsen et al, Chembiochem, 2015, 16(16), 2289-93; Justyna et al., Current Drug Targets, 2013, 14(7), 798-816)). Rats were surgically implanted with a jugular vein catheter (sterile polyethylene, o.d. 0.96 mm i.d. 0.58 mm, Microtube Extrusions, Aust.) and allowed to recover overnight. Baseline blood samples (t=0) were taken prior to peptide administration directly from the indwelling jugular vein catheter. Peptide was administered directly via the catheter (i.v route), followed by a 200 μL chaser volume of heparinised saline (20 U/mL) to flush the line. Further blood samples (200 μL/sample) were collected directly from the intravenous catheter at t=1, 5 15, 30, 60, 90 120 as 180 minutes post-administration. Volumes taken were replaced with heparinised saline to avoid hypovolaemia. Blood samples were immediately centrifuged (14 k rpm, 5 min) and plasma supernatant snap frozen on dry ice for later processing. Plasma samples were thawed and diluted 1 in 4 acetonitrile, vortexed, sonicated and centrifuged. Supernatants were transferred to HPLC vials for LCMS analysis.

A Triple Quad Mass-Spectrometer (model 6460, Agilent Technologies) coupled to a Bidentate C18 HPLC column (Cogent, 100 Å, 4 μM) was used to analyse all samples. Separations were carried out using a binary solvent gradient. A Multiple Reaction Monitoring (MRM) protocol was developed for each individual peptide to maximise sensitivity of the Mass-spec and optimise the Limit of Detection (LOD) from a serially diluted standard curve, performed in both acetonitrile solvent and neat rat plasma.

Samples were analysed using MassHunter Software packages, and area under the curve (AUC) was measured for each peak of interest in the MRM, as a direct measure of concentration of compound ions in the sample. AUCs were converted to plasma concentrations (Cₚ; μg/mL and μM) using the standard curves generated in plasma. Data was analysed using MS Excel software. A two compartment model was adopted to determine the pharmacokinetic properties of each peptide in the rat. The first phase describes the distribution throughout the circulatory compartment, and the second phase describes the elimination/steady state concentration in plasma/tissue. Plasma half-life, volume of distribution and clearance were determined from the steady-state elimination phase.

The peptides tested were SEQ ID Nos 19 and 24. Plasma concentrations (Cₚ) were plotted against time and the PK parameters determined (Table 8). All three compounds had a predictably short half-life (t_{1/2}<30 min) common to similar peptides. Initially the peptides were administered at the lowest maximal effective dose given in the FCA experiments (0.3 mg/kg), with the concern of KOR-mediated side effects/over dose posing risk to the experimental animals' welfare. However, SEQ ID NO 19 showed no sign of opioid-like adverse effects (e.g. respiratory depression, sedation) at this dose. Unfortunately, detection of the peptides in plasma samples at latter time point after administration was difficult since Cₚ was lower than the apparent LOD. Hence SEQ ID NO 24 was administered at 1 mg/kg i.v. with hope of increasing the potential to detect at latter time points whilst still avoiding potential adverse effects. No adverse effects were observed. Increasing dose did not improve the Cₚ as anticipated.

TABLE 8

| | Dose | | Extract | | | |
|---|---|---|---|---|---|---|
| | (mg/kg, | LOD | efficiency | V_{dss} | T_{1/2} | CLₜₒₜₐₗ |
| peptide | i.v.) | (nM) | (%) | (L/kg) | (min) | (mL/min/kg) |
| SEQ ID | 0.3 | 10 | 54.4 | 0.4 | 23.9 | 5.7 |
| NO. 19 | | | | | | |
| SEQ ID | 1 | 10 | 62.4 | 1.7 | 12.8 | 26.3 |
| NO. 24 | | | | | | |

SEQ ID NO 24 also showed a rapid clearance (CLₜₒₜₐₗ). Although, here total clearance is measured, which is (among other effects) subject to tissue partitioning affecting the detectable plasma concentrations, and does not necessarily relate to excretion. SEQ ID NO 19 showed the lowest clearance. SEQ ID NO 24 was the shortest lived in plasma, both in half-life and clearance.

The volume of distribution (V_{dss}) of SEQ ID NO. 19 was relatively low, suggesting the peptide was confined to total body water. SEQ ID NO 24 has a V_{dss} of greater than 1.0, which suggests some degree of tissue distribution. The relatively large difference in V_{dss} between SEQ ID NO 19 (V_{dss} 0.4 L/kg) and SEQ ID NO 24 (V_{dss} 1.7 L/kg) is not surprising even though both are very similar linear peptides. SEQ ID NO 24 carries a lipidated component, which in theory will render it more membrane permeable and therefore gives it a greater propensity to partition to tissue compartments. The low extraction efficiency, the low LOD, the low Cₚ (even with increased dose of SEQ ID NO. 24), and the initial V_{d} (at t=0, not reported), all are crudely indicative of a high degree of plasma protein binding (PPB). Actual measurements of PPB were not made.

1. A compound of formula (I), or a pharmaceutically acceptable salt, solvate, stereoisomer or prodrug thereof:
R₁NH—X₁—X₂—X₃—X₄—X₅—X₆—X₇—X₈—X₉—X₁₀—X₁₁—C(O)R₂ (I)
wherein R₁ is selected from the group consisting of hydrogen and C₁₋₆alkyl;
R₂ is selected from the group consisting of OH, NH₂, NH(C₁₋₆alkyl) and N(C₁₋₆alkyl)₂;
X₁ is selected from the group consisting of L-tyrosine, D-tyrosine, L-phenylalanine, D-phenylalanine, L-3-(4-pyridyl)-alanine and D-3-(4-pyridyl)-alanine, a tyrosine derivative, a phenylalanine derivative and a 3-(4-pyridyl)-alanine derivative;
X₂ is selected from the group consisting of glycine, sarcosine, N-alkylglycine, 4-aminobutyric acid, L-leucine, D-leucine, L-isoleucine, D-isoleucine, L-valine, D-valine, L-alanine, D-alanine, L-3-(4-pyridyl)-alanine and D-3-(4-pyridyl)-alanine;
X₃ is absent;
X₄ is selected from the group consisting of L-phenylalanine, D-phenylalanine, L-leucine, D-leucine, a phenylalanine derivative and a leucine derivative;
X₅ is selected from the group consisting of glycine, L-leucine, D-leucine, L-isoleucine, D-isoleucine, L-valine and D-valine;
X₆ is selected from the group consisting of a positively charged amino acid residue, a negatively charged amino acid residue and a polar uncharged amino acid residue;
X₇ is selected from the group consisting of a positively charged amino acid residue, a negatively charged amino acid residue and a polar uncharged amino acid residue;
X₈ is absent or is selected from the group consisting of a hydrophobic amino acid residue and —C₁₋₁₀ alkylene-;
X₉ is absent or is selected from the group consisting of a positively charged amino acid residue and a polar uncharged amino acid residue;
X₁₀ is absent or is a hydrophobic amino acid residue; and
X₁₁ is absent or is a positively charged amino acid residue;
wherein at least one amino acid residue X₁, X₂ and X₄ to X₇ is a non-proteinogenic amino acid;
or a pharmaceutically acceptable salt, solvate, stereoisomer or prodrug thereof.

2. The compound according to claim 1 wherein a non-proteinogenic amino acid is located at X₄.

3. The compound according to claim 1 wherein all of X₁, X₂ and X₄ to X₁₁ are in the L-configuration.

4. The compound according to claim 1 wherein one to three of X₁, X₂ and X₄ to X₇ are in the D-configuration.

5. The compound according to claim 1, wherein R₁ is hydrogen or methyl.

6. The compound according to claim 1, wherein R₂ is OH or NH₂.

7. The compound according to claim 1, wherein X₁ is L-tyrosine, phenylalanine or L-3-(4-pyridyl)-alanine.

8. (canceled)

9. The compound according to claim 1, wherein X₂ is glycine, sarcosine, γ-aminobutyric acid, L-alanine, or L-3-(4-pyridyl)-alanine.

10. (canceled)

11. (canceled)

12. The compound according to claim 1, wherein X₄ is L-phenylalanine, D-phenylalanine or a phenylalanine derivative.

13. (canceled)

14. The compound according to claim 1, wherein X₅ is L-leucine.

15. The compound according to claim 1, wherein X₆ is L-lysine, L-arginine, L-histidine, L-ornithine, D-lysine, D-arginine, D-histidine, D-ornithine, N-methyl-L-lysine, N-methyl-L-arginine, N-methyl-L-histidine, N-methyl-L-ornithine, N-methyl-D-lysine, N-methyl-D-arginine, N-methyl-D-histidine, N-methyl-D-ornithine, L-diaminobutyric acid, D-diaminobutyric acid, N-methyl-L-diaminobutyric acid, N-methyl-D-diaminobutyric acid, L-citrulline, D-citrulline, N-methyl-L-citrulline, N-methyl-D-citrulline, L-homoarginine, D-homoarginine, N-methyl-L-homoarginine, N-methyl-D-homoarginine, L-asparagine, L-glutamine, D-asparagine, D-glutamine, N-methyl-L-asparagine, N-methyl-L-glutamine, N-methyl-D-asparagine or N-methyl-D-glutamine.

16. (canceled)

17. The compound according to claim 1, wherein X₇ is L-lysine, L-arginine, L-histidine, L-ornithine, D-lysine, D-arginine, D-histidine, D-ornithine, N-methyl-L-lysine, N-methyl-L-arginine, N-methyl-L-histidine, N-methyl-L-ornithine, N-methyl-D-lysine, N-methyl-D-arginine, N-methyl-D-histidine, N-methyl-D-ornithine, L-diaminobutyric acid, D-diaminobutyric acid, N-methyl-L-diaminobutyric acid, N-methyl-D-diaminobutyric acid, L-citrulline, D-citrulline, N-methyl-L-citrulline, N-methyl-D-citrulline, L-homoarginine, D-homoarginine, N-methyl-L-homoarginine, N-methyl-D-homoarginine, L-4,4-carboxyaminopiperidine, D-4,4-carboxyaminopiperi dine, L-N-Methyl-4,4-carboxyaminopiperidine, D-N-Methyl-4,4-carboxyaminopiperidine, L-asparagine, L-glutamine, D-asparagine, D-glutamine, N-methyl-L-asparagine, N-methyl-L-glutamine, N-methyl-D-asparagine or N-methyl-D-glutamine.

18. (canceled)

19. (canceled)

20. The compound according to claim 1, wherein X₈ is absent or is —C₄₋₈alkylene- or L-isoleucine.

21. The compound according to claim 1, wherein X₉ is absent or is L-arginine or D-arginine.

22. The compound according to claim 1, wherein X₁₀ is absent or is L-proline.

23. The compound according to claim 1, wherein X₁₁ is absent or is L-lysine.

24. The compound according to claim 1 selected from the group consisting of:

| SEQ | |
|---|---|
| ID | |
| NO. | Sequence |
| 1 | NH₂-Tyr-Gaba-Phe-Leu-Arg-Arg-NH₂ |
| 2 | NH₂-Tyr-Sar-Phe-Leu-Arg-Arg-NH₂ |
| 3 | NH₂-Tyr-Sar-Phe-Leu-Arg-DArg-NH₂ |
| 4 | NH₂-Tyr-Sar-(4-NO₂)Phe-Leu-Arg-DArg-NH₂ |
| 5 | NH₂-Tyr-Sar-(4-NO₂)Phe-Leu-DArg-Arg-NH₂ |
| 6 | NH₂-Tyr-Sar-Phe-Leu-DArg-Arg-NH₂ |
| 7 | NH₂-Tyr-Sar-Phe-Leu-DArg-DArg-NH₂ |
| 8 | NH₂-Tyr-Gly-(4-NO₂)Phe-Leu-DArg-DArg-NH₂ |
| 9 | NH₂-Phe-Sar-(4-NO₂)Phe-Leu-DArg-DArg-NH₂ |
| 10 | NH₂-Tyr-Sar-(4-NO₂)Phe-Leu-DLys-DArg-NH₂ |
| 11 | NH₂-Tyr-Sar-(4-C1)Phe-Leu-Arg-DArg-NH₂ |
| 12 | NH₂-Tyr-Sar-(4-F)Phe-Leu-Arg-DArg-NH₂ |
| 13 | NH₂-Tyr-Sar-(4-Cl)Phe-Leu-NMA-DArg-NH₂ |
| 14 | NH₂-Tyr-Sar-(4-F)Phe-Leu-NMA-DArg-NH₂ |
| 15 | NH₂-Tyr-Sar-(4-NO₂)Phe-Leu-NMA-DArg-NH₂ |
| 16 | NH₂-Tyr-Sar-(4-C1)Phe-Leu-Lys-DArg-NH₂ |
| 17 | NH₂-Tyr-Sar-(4-C1)Phe-Leu-Arg-NMA-NH₂ |
| 18 | NH₂-Tyr-Sar-(4-NO₂)Phe-Leu-Arg-NMA-NH₂ |
| 19 | NH₂-Tyr-Sar-(4-NO₂)Phe-Leu-NMA-NMA-NH₂ |
| 20 | NH₂-Tyr-Sar-(4-C1)Phe-Leu-Arg-DArg-Ile- |
| | Arg-NH₂ |
| 21 | NH₂-Tyr-Sar-(4-C1)Phe-Leu-Arg-DArg-C₅H₁₀- |
| | CONH₂ |
| 22 | NH₂-Tyr-Sar-(4-Cl)Phe-Leu-Arg-DArg-Ile-Arg- |
| | Pro-Lys-NH₂ |
| 23 | NH₂-Tyr-Sar-(4-Cl)Phe-Leu-Arg-DArg-Ile-Arg- |
| | Pro-Dlys-NH₂ |
| 24 | NH₂-Tyr-Sar-(4-Cl)Phe-Leu-Arg-DArg-C₇H₁₄- |
| | CONH₂ |
| 25 | NH₂-Tyr-Sar-(4-Cl)Phe-Leu-Arg-DArg-Ile- |
| | DArg-NH₂ |
| 26 | NH₂-Tyr-Sar-(4-Cl)Phe-Leu-Arg-NMA-Ile-Arg- |
| | NH₂ |
| 27 | NH₂-Tyr-Sar-(4-NO₂)Phe-Leu-Arg-NMA-Ile-Arg- |
| | NH₂ |
| 28 | NH₂-Tyr-Sar-(4-Cl)Phe-Leu-Arg-DArg-C₇H₁₄- |
| | COOH |
| 29 | NH₂-Tyr-Sar-(4-Cl)Phe-Leu-Arg-NMA-C₇H₁₄- |
| | CONH₂ |
| 30 | NH₂-Tyr-Sar-(4-NO₂)Phe-Leu-Arg-NMA-C₇H₁₄- |
| | CONH₂ |
| 31 | NH₂-Tyr-Sar-(4-NO₂)Phe-Leu-Arg-DArg-C₇H₁₄- |
| | CONH₂ |

25. A pharmaceutical composition comprising a compound of formula (I) according to claim 1 or pharmaceutically acceptable salt, solvate, stereoisomer or prodrug thereof and a pharmaceutically acceptable carrier, diluent and/or excipient.

26. A method of treating or preventing pain in a subject in need thereof, comprising administering an effective amount of a compound of formula (I) according to claim 1 or a pharmaceutically acceptable salt, solvate, stereoisomer or prodrug thereof, or a pharmaceutical composition comprising a compound of formula (I) according to claim 1 or a pharmaceutically acceptable salt, solvate, stereoisomer or prodrug thereof.

27. (canceled)

28. (canceled)

29. The method of claim 26, wherein the pain is acute pain, chronic pain, nociceptive pain, inflammatory pain or neuropathic pain.

30. (canceled)
